(19) United States
(12) Patent Application Publication
(10) Pub. No.: US 2020/0188888 A1
(43) Pub. Date: Jun. 18, 2020

(54) MESOPOROUS SILICA SUPPORTED CATALYST FOR OXIDATIVE DEHYDROGENATION

(71) Applicant: King Fahd University of Petroleum and Minerals, Dhahran (SA)

(72) Inventors: Palani Aruda, Dhahran (SA); Sulaiman Saleh Al-Khattaf, Dhahran (SA); Sachio Asaoka, Dhahran (SA)

(73) Assignee: King Fahd University of Petroleum and Minerals, Dhahran (SA)

(21) Appl. No.: 16/218,587

(22) Filed: Dec. 13, 2018

(51) Int. Cl. B01J 23/843; B01J 35/10; C07C 5/48; C07C 11/08

(52) U.S. Cl. CPC B01J 23/8437; B01J 35/1038; B01J 35/1042; B01J 35/1047; B01J 35/1061; C07C 2523/843; B01J 35/1023; C07C 5/48; C07C 11/08; B01J 2523/54; B01J 2523/847; B01J 35/1019

(57) ABSTRACT

Oxidative dehydrogenation catalysts comprising bismuth and nickel oxides impregnated on mesoporous silica supports such as SBA-15 and mesoporous silica foam. Methods of preparing and characterizing the catalysts as well as processes for oxidatively dehydrogenating n-butane to butadiene using the catalysts are also described. The disclosed catalysts demonstrate higher n-butane conversion and butadiene selectivity than catalysts supported by conventional silica.

### STATEMENT REGARDING PRIOR DISCLOSURE BY THE INVENTORS

Aspects of this technology are described in an article “Pore structure effect of support in Ni—Bi—O/mesoporous silica catalyst on oxidative dehydrogenation of n-butane to butadiene” published in Catalysis Today on Jun. 8, 2018, which is incorporated herein by reference in its entirety.

### BACKGROUND OF THE INVENTION

### Technical Field

The present invention relates to mesoporous silica supported bismuth and nickel oxides based catalysts and their use for oxidative dehydrogenation of n-butane to butadiene.

### Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Recently there has been a growing demand for butadiene, which is an important raw material for the petrochemical industry. Butadiene is mainly produced as a byproduct from cracking naphtha to form ethylene and propylene. “On-purpose” butadiene production technologies are expected to supply the growing market. One of these technologies is oxidative dehydrogenation of 1-butene.

Another option is oxidative dehydrogenation of n-butane to butadiene however an efficient catalyst system is required. The CATADIENE® dehydrogenation process is a commercialized route for the production of butadiene from n-butane or a mix of n-butane and n-butenes. UOP has partnered with TPC Group to produce “on-purpose” butadiene from n-butene using OXO-D™ technology.

Stoimenov, Peter K and Sherman, Jeffrey H [WO Patent Application No. 2015/191580 A1, incorporated herein by reference in its entirety] discloses a method of using n-butane and bromine in brominating reactors to form a bromination product then subjecting the product to dehydrobromination thereby forming a dehydrobromination product stream comprising 1,3-butadiene. The process may be implemented for selective bromination of butene at an allylic position to form a bromination product using butene and bromine under adapted reaction conditions and subjecting the bromobutenes to dehydrobromination to form a dehydrobromination product stream comprising 1,3-butadiene. Stoimenov also discloses a method of forming an oxybromination product stream comprising bromobutenes by mixing butene with hydrogen bromide in the presence of oxygen in an oxybromination reactor under sufficient reaction conditions, and subjecting the bromobutenes to dehydrobromination thereby forming a dehydrobromination product stream comprising 1,3-butadiene.

Hiraoka, Ryota et al. [WO Patent Application No. 2013/161702 A1, incorporated herein by reference in its entirety] discloses a catalyst for the production of butadiene using n-butane as the starting material. A molybdenum mixed oxide composite having a formula of MoₐBi_{b}Ni_{c}Co_{d}Fe_{f}X_{g}YₕOₓ (X=tungsten, antimony, etc.; Y=potassium, rubidium, etc.; a=12; b=0.1-7; c+d=0.5-20; f=0.5-8; g=0-2; h=0.005-2; and xis defined in accordance with oxidation states of each element) containing micropores was used as the catalyst.

Rabindran Jermy et al. [Journal of Molecular Catalysis A: Chemical 400 (2015) 121-131, incorporated herein by reference in its entirety] discloses oxidative dehydrogenation of n-butane to 1,3-butadiene over Bi—Ni oxide/-Al₂O₃ catalyst with a fixed bed/flow-type reactor at 400-500° C. A nickel oxide loading of 30 wt % Ni on Al₂O₃ resulted higher catalytic activity and selectivity of butadiene as a result of diminished partial oxidation related to Al₂O₃ and enhanced conversion to butadiene. A catalyst having 30 wt % Bi and 20 wt % Ni oxide over Al₂O₃ showed the highest butadiene selectivity of about 47% at 450° C.

Tanimu et al. [Molecular Catalysis 438 (2017) 245-255, incorporated herein by reference in its entirety] discloses catalyst containing 20 wt % Ni and 30 wt % Bi (by metal weight) supported on Al₂O₃, SiO₂ (sol and gel types), or ZrO₂ for oxidative dehydrogenation of n-butane to butadiene. The activity and selectivity of oxidative dehydrogenation of n-butane to butadiene over the Ni—Bi—O/support catalyst strongly depended on the identity of support. The order of catalytic activity and butadiene selectivity of catalysts having sol-type support is SiO₂>Al₂O₃>ZrO₂. SiO₂ sol shows superior performance over SiO₂ gel-type. Additionally, SiO₂ sol supported catalyst, which has n-butane conversion at 35.6%, selectivity of dehydrogenation of 78.3%, and selectivity of butadiene of 41.6%, is the best performing among all catalysts studied. A balanced number of acid and base sites in the sol-type SiO₂ as well as suppressed oxygenate by the accelerating redox system with non-hierarchical nanoparticle cohabitation of NiO, Bi₆O₇ and SiO₂ support contributed to the butadiene selectivity.

Catalysts fixed on mesoporous silica for the production of butadiene from butane have not been disclosed or studied. Various loadings of Ni and Bi oxides impregnated on different types of mesoporous silica (MCM-41, SBA-15 and silica foam) provide an option for testing catalytic activity in the production of butadiene from butane.

In view of the forgoing, one objective of the present disclosure is to provide catalysts comprising nickel and bismuth oxides impregnated on a mesoporous silica support including SBA-15 and/or mesoporous silica foam. A further objective of the present disclosure is to provide a method of producing these catalysts. An additional objective of the present disclosure is to provide a process of oxidatively dehydrogenating n-butane to butadiene employing these catalysts which leads to enhanced n-butane conversion and butadiene selectivity.

### BRIEF SUMMARY OF THE INVENTION

According to a first aspect, the present disclosure relates to a catalyst including a mesoporous silica support which is at least one selected from the group consisting of SBA-15 and mesoporous silica foam, and a catalytic material comprising nickel oxide and bismuth oxide impregnated on the mesoporous silica support, wherein the nickel and bismuth atoms of the nickel oxide and the bismuth oxide are present in amounts of 2-30 wt % and 5-40 wt %, each relative to a weight of the mesoporous silica support.

In one embodiment, the mesoporous silica support has a pore volume of 0.2-3 cm³/g and a BET surface area of 200-1,000 m²/g.

In one embodiment, the mesoporous silica support is SBA-15.

In one embodiment, 50-99.9 wt % of the bismuth oxide is present as a non-stoichiometric bismuth oxide relative to a total weight of the bismuth oxide.

In one embodiment, the non-stoichiometric bismuth oxide has a formula of Bi₂O₃₋ₓ in which x ranges from 0.2 to 0.4.

In one embodiment, the catalyst has an average pore diameter of 2-20 nm.

In one embodiment, the catalyst has a pore volume of 0.2-2 cm³/g.

In one embodiment, the catalyst has a BET surface area of 200-700 m²/g.

According to a second aspect, the present disclosure relates to a method of preparing the catalyst of the first aspect, in one or more of its embodiments. The method involves (i) mixing the mesoporous silica support with an aqueous solution comprising a nickel salt and a bismuth salt to form a mixture, (ii) drying the mixture to form a dried mass, and (iii) calcining the dried mass in air at a temperature of 300-700° C., thereby producing the catalyst.

In one embodiment, a weight ratio of the mesoporous silica support to the nickel salt ranges from 1:3 to 3:1, and a weight ratio of the mesoporous silica support to the bismuth salt ranges from 1:2 to 4:1.

In one embodiment, the aqueous solution comprises the nickel salt at a concentration of 20-80 mM, and the bismuth salt at a concentration of 10-40 mM.

According to a third aspect, the present disclosure relates to a process of oxidatively dehydrogenating n-butane to form butadiene and butenes, the process involving flowing a feed mixture comprising n-butane and an oxidant through a reactor loaded with the catalyst of the present disclosure, in one or more of its embodiments, thereby forming butadiene and butenes.

In one embodiment, a molar ratio of the oxidant to n-butane is in a range of 0.1:1 to 8:1.

In one embodiment, the feed mixture is flowed at a flow rate of 10-100 mL/min.

In one embodiment, flowing is performed at a temperature of 300-700° C.

In one embodiment, flowing is performed at a pressure of 35-350 kPa.

In one embodiment, the oxidant is O₂.

In one embodiment, the feed mixture further comprises an inert gas.

In one embodiment, the process of the third aspect has a butadiene yield of 7-40 wt % relative to a weight of n-butane.

In one embodiment, the process of the third aspect has a molar ratio of butadiene to butenes in a range of about 1:1 to about 4:1.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is an overlay of X-ray diffraction (XRD) patterns of different catalysts each including a silica support (A: Cariact Q-6, B: Cariact Q-10, C: Cariact Q-30, D: SBA-15, E: MCM-41, and F: mesoporous silica foam) and a catalytic material of nickel oxide and bismuth oxide impregnated on the respective silica support.

FIG. 2 shows a possible reaction pathway for oxidative dehydrogenation of n-butane to butadiene.

FIG. 3 is a comparison of XRD patterns of NiO/SBA-15 (20 wt % Ni), BiOₓ/SBA-15 (30 wt % Bi), and catalyst D.

FIG. 4 is a comparison of Bi₂O₃₋ₐ phase concentrations in mesopore and conventional support pore diameter.

FIG. 5A shows pore size distributions of MCM-41, silica foam, and SBA-15, respectively.

FIG. 5B shows pore size distributions of catalysts D, E, and F, respectively.

FIG. 5C shows pore size distributions of supports Cariact Q6, Cariact Q10 and Cariact Q30, respectively.

FIG. 5D shows pore size distribution of catalysts A, B, and C, respectively.

FIG. 6 is an overlay of temperature programmed reduction (TPR) profiles of different support species with Ni—Bi—O catalyst: B (Cariact Q10), D (Si-SBA-15), E (Si-MCM-41), and F (SiO₂ foam).

FIG. 7A is a transmission electron microscope (TEM) image of catalyst E.

FIG. 7B is a TEM image of catalyst D.

FIG. 7C is a TEM image of catalyst F.

FIG. 8 is a high resolution transmission electron microscope (HRTEM) image of catalyst D.

FIG. 9A is a graph showing n-butane conversions of different catalysts A-F.

FIG. 9B is a graph showing butadiene selectivity of different catalysts A-F.

FIG. 10A is a graph showing the relationship between n-butane conversion and Bi₂O₃₋ₐ (a=0.2-0.4) phase concentration in mesopore.

FIG. 10B is a graph showing the relationship between butadiene selectivity and Bi₂O₃₋ₐ (a=0.2-0.4) phase concentration in mesopore.

FIG. 11 is a graph showing the relationship between butadiene yield and Bi₂O₃₋ₐ phase concentration in mesopore.

FIG. 12 is an illustration of models of reaction and catalyst for oxidative dehydrogenation of n-butane to butadiene over Bi—Ni oxide/SiO₂ catalyst to show the effect of support on butadiene (C₄⁼) selectivity from n-butane (C₄⁰) over hierarchical nanoparticle cohabitation catalysts.

### DETAILED DESCRIPTION OF THE EMBODIMENTS

Embodiments of the present disclosure will now be described more fully hereinafter with reference to the accompanying drawings, in which some, but not all embodiments of the disclosure are shown.

The present disclosure may be better understood with reference to the following definitions. As used herein, the words “a” and “an” and the like carry the meaning of “one or more.” Within the description of this disclosure, where a numerical limit or range is stated, the endpoints are included unless stated otherwise. Also, all values and subranges within a numerical limit or range are specifically included as if explicitly written out.

As used herein, the words “substantially similar”, “approximately”, or “about” may be used when describing magnitude and/or position to indicate that the value and/or position described is within a reasonable expected range of values and/or positions. For example, a numeric value may have a value that is ±1% of the stated value (or range of values), ±2% of the stated value (or range of values), ±5% of the stated value (or range of values), ±10% of the stated value (or range of values), ±15% of the stated value (or range of values), or ±20% of the stated value (or range of values).

As used herein, the term “compound” refers to a chemical entity, whether in a solid, liquid or gaseous phase, and whether in a crude mixture or purified and isolated.

The present disclosure includes all hydration states of a given compound or formula, unless otherwise noted. For example, Ni(NO₃)₂ includes anhydrous Ni(NO₃)₂, Ni(NO₃)₂.6H₂O, and any other hydrated forms or mixtures. Bi(NO₃)₃ includes both anhydrous Bi(NO₃)₃ and Bi(NO₃)·5H₂O.

The present disclosure further includes all isotopes of atoms occurring in the present compounds. Isotopes include those atoms having the same atomic number but different mass numbers. By way of general example, and without limitation, isotopes of hydrogen include deuterium and tritium, isotopes of carbon include ¹³C and ¹⁴C, isotopes of oxygen include ¹⁶O, ¹⁷O and ¹⁸O, and isotopes of nickel include ⁵⁸Ni, ⁶⁰Ni, ⁶¹Ni, and ⁶⁴Ni. Isotopically labeled compounds of the disclosure can generally be prepared by conventional techniques known to those skilled in the art or by processes and methods analogous to those described herein, using an appropriate isotopically labeled reagent in place of the non-labeled reagent otherwise employed.

According to a first aspect, the present disclosure relates to a catalyst including a mesoporous silica support which is at least one selected from the group consisting of mesoporous silica such as SBA-15 and mesoporous silica foam, and a catalytic material comprising nickel oxide and bismuth oxide impregnated on the mesoporous silica support.

As used herein, a support material refers to a material, usually a solid with a surface area, to which a catalyst is affixed. A “mesoporous support” refers to a porous support material with largest pore diameters ranging from about 2-50 nm, preferably 3-45 nm, preferably 4-40 nm, preferably 5-25 nm. Typical support materials include various kinds of carbon, alumina, and silica. In a preferred embodiment, the catalyst of the present disclosure comprises a mesoporous silica support. In one or more embodiments, the mesoporous silica support is at least one selected from the group consisting of SBA-15 and mesoporous silica foam. In a preferred embodiment, the mesoporous silica support is SBA-15.

The Brunauer-Emmet-Teller (BET) theory (S. Brunauer, P. H. Emmett, E. Teller, J. Am. Chem. Soc. 1938, 60, 309-319, incorporated herein by reference) aims to explain the physical adsorption of gas molecules on a solid surface and serves as the basis for an important analysis technique for the measurement of a specific surface area of a material. Specific surface area is a property of solids which is the total surface area of a material per unit of mass, solid or bulk volume, or cross sectional area. In most embodiments, pore volume and BET surface area are measured by gas adsorption analysis, preferably N₂ adsorption analysis.

In one or more embodiments, the mesoporous silica support has a pore volume of 0.2-3 cm³/g, 0.5-2.5 cm³/g, 0.8-2.0 cm³/g, or 1.0-1.5 cm³/g. In one or more embodiments, the mesoporous silica support has a BET surface area of 200-1,000 m²/g, 300-800 m²/g, 400-700 m²/g, or 500-600 m²/g.

In one embodiment, the mesoporous silica support has pore channels that are regularly arranged. For example, the mesoporous silica support is in the form of a honeycomb-like structure having pore channels parallel or substantially parallel to each other within a two-dimensional hexagon (e.g. SBA-15). Alternatively, other mesoporous silica structures of the SBA series such as SBA-11 having a cubic structure, SBA-12 having a three-dimensional hexagonal structure, and SBA-16 having a cubic in cage-like structure may be used as the mesoporous silica support. In one embodiment, the mesoporous silica support is in the form of SBA-15, and the mesoporous silica support has a pore volume of 0.2-1.5 cm³/g, 0.4-1.2 cm³/g, or 0.5-1.0 cm³/g and a BET surface area of 200-800 m²/g, 400-700 m²/g, or 500-600 m²/g.

As defined herein, a silica foam is a foam silicate that has interconnected cells joined at a nexus, with varying properties depending on the nature of the surfactant used and the method of synthesis. A surfactant templated mesoporous silica foam has porosity resulting from the presence of silicate struts that define cage-like cellular pores connected by windows. In one embodiment, the mesoporous silica support described herein is a mesoporous silica foam. In certain embodiments, the mesoporous silica foam is amorphous having a non-ordered structure. This non-ordered structure may be random and thus different than the aforementioned mesoporous silica structures and SBA silica structures. Specifically, when a mesoporous silica foam is used, the mesoporous silica support has a pore volume of 1.5-3 cm³/g, 2.0-2.7 cm³/g, or 2.2-2.5 cm³/g, and a BET surface area of 400-1,000 m²/g, 500-800 m²/g, or 600-700 m²/g.

Nickel atoms of the nickel oxide may be present in an amount of 2-30 wt %, preferably 5-26 wt %, more preferably 10-21 wt %, or about 20 wt % relative to a total weight of the mesoporous silica support. Bismuth atoms of the bismuth oxide are present in an amount of 5-40 wt %, preferably 10-36 wt %, more preferably 20-31 wt %, or about 30 wt % relative to a weight of the mesoporous silica support. The amounts of nickel and bismuth atoms in the catalyst may vary depending upon the properties sought (e.g. catalytic activities, surface property of the mesoporous silica support) as well as the dispersibility of nickel and bismuth oxides in the mesoporous silica support. In some embodiments, nickel oxide is present in the catalyst at an amount of 2-40 wt %, 6-35 wt %, or 12-28 wt % relative to a weight of the mesoporous silica support. In some embodiments, bismuth oxide is present in the catalyst at an amount of 5-45 wt %, 10-40 wt %, or 24-35 wt % relative to a weight of the mesoporous silica support.

In one embodiment, the bismuth oxide may be comprised of a plurality of different crystallographic phases. Bismuth oxide (Bi₂O₃) has five crystallographic polymorphs. The room temperature phase, α-Bi₂O₃ is stable and has a monoclinic crystal structure having layers of oxygen atoms with layers of bismuth atoms between them. There are three high temperature phases, a tetragonal β-phase, a body-centered cubic γ-phase, a cubic δ-Bi₂O₃ phase and a ε-phase. In the present disclosure, the bismuth oxide may refer to Bi₂O₃ having an α polymorph, a β polymorph, a γ polymorph, a δ polymorph, a ε polymorph, or mixtures thereof. In another embodiment, the bismuth oxide may be amorphous, or have different crystal morphology. As used herein, a non-stoichiometric bismuth oxide (Bi₂O₃) refers to a bismuth oxide having its Bi:O ratio deviated from 2:3. Non-stoichiometric bismuth oxides may include crystal lattice defects such as metal or oxygen vacancies and may contain mixed valent bismuth, i.e., Bi(0), Bi(II), Bi(III) and/or Bi(V) species.

X-ray diffraction (XRD) patterns of the bismuth oxide may provide information including, which crystalline phases are present (peak locations), relative amounts of each phase (integrated area under respective peaks), and crystallite size (peak width at half max). The different bismuth oxide phases as well as non-stoichiometric species that can be present in the mesoporous silica support may depend on the synthesis method, the bismuth precursor, solvent, calcination temperature, bismuth oxide loading, the mesoporous silica support, etc. In one embodiment, 50-99.9 wt %, preferably 80-99.5 wt %, more preferably 90-95 wt % of the bismuth oxide is present as a non-stoichiometric bismuth oxide relative to a total weight of the bismuth oxide. In certain embodiment, the bismuth oxide comprises a non-stoichiometric bismuth oxide in terms of its oxygen vacancy (e.g. Bi₂O₃₋ₓ). In a preferred embodiment, the non-stoichiometric bismuth oxide has a formula of Bi₂O₃₋ₓ, in which x ranges from 0.1-0.7, preferably 0.15-0.6, more preferably 0.2 to 0.4.

In one embodiment, the nickel oxide may have a NiO crystalline bunsenite morphology, which has an isometric or cubic crystal system. The crystalline NiO may have a structure within the Fm3m space group, and the structure may be part of the hexoctahedral crystal class. In another embodiment, the nickel oxide may be amorphous, or have different crystal morphology. In certain embodiments, the nickel oxide comprises less than 100 wt % NiO, and further comprises Ni(0) and/or Ni(III) (e.g. Ni₂O₃). Preferably the nickel oxide comprises at least 90 wt % NiO, preferably at least 95 wt % NiO, more preferably at least 99 wt % NiO, even more preferably 99.5 wt % NiO, or about 100 wt % NiO, relative to a total weight of the nickel oxide.

In a preferred embodiment, the catalyst of the present disclosure comprises a catalytic material comprising nickel oxide and bismuth oxide impregnated on a mesoporous silica support. As used herein, “impregnated” or “disposed on” describes being completely or partially filled throughout, saturated, permeated, and/or infused. The catalytic material may be affixed on one or more surfaces of the mesoporous silica support. For example, the catalytic material may be affixed on an outer surface of the mesoporous silica support or within pore spaces of the mesoporous silica support. The catalytic material may be affixed to the mesoporous silica support in any reasonable manner, such as physisorption, chemisorption, or combinations thereof. In one embodiment, greater than 10% of the surface area (i.e. outer surface and pore spaces) of the mesoporous silica support is covered by the catalytic material. Preferably greater than 15%, preferably greater than 20%, preferably greater than 25%, preferably greater than 30%, preferably greater than 35%, preferably greater than 40%, preferably greater than 45%, preferably greater than 50%, preferably greater than 55%, preferably greater than 60%, preferably greater than 65%, preferably greater than 70%, preferably greater than 75%, preferably greater than 80%, preferably greater than 85%, preferably greater than 90%, preferably greater than 95%, preferably greater than 96%, preferably greater than 97%, preferably greater than 98%, or preferably greater than 99% of the surface area of the mesoporous silica support is covered by the catalytic material.

In one embodiment, the mesoporous silica support is covered with a thin layer of the catalyst material having an average thickness of 0.5-50 nm, 1-45 nm, 5-40 nm, 10-35 nm, or 20-30 nm. At high enough loadings, nanocrystals or nanoparticles of the catalytic material (e.g. bismuth oxide, nickel oxide, and mixtures thereof) having an average particle size of 1-500 nm, preferably 5-400 nm, preferably 10-300 nm, preferably 20-200 nm, preferably 40-100 nm, preferably 50-75 nm may be present on the surface of the mesoporous silica support. In one or more embodiments, the nickel and bismuth oxides are homogeneously distributed throughout the mesoporous silica support. In a preferred embodiment, the nickel oxide is dispersed on the bismuth oxide. Alternatively, the nickel oxide may form localized clusters amongst the bismuth oxide. In certain embodiments, the different bismuth and nickel species and their distributions on the mesoporous silica support may be identified by techniques including, but not limited to, UV-vis spectroscopy, XRD, Raman spectroscopy, AFM (atomic force microscope), TEM (transmission electron microscopy), and EPR (electron paramagnetic resonance).

A particle is defined as a small object that behaves as a whole unit with respect to its transport and properties. The catalyst of the present disclosure in any of its embodiments may be in the form of particles of the same shape or different shapes, and of the same size or different sizes. An average diameter (e.g., average particle diameter) of the particle, as used herein, refers to the average linear distance measured from one point on the particle through the center of the particle to a point directly across from it. The catalyst particles may have an average diameter in a range of 5-800 nm, 10-600 nm, 20-400 nm, 30-300 nm, 40-200 nm, or 50-100 nm. In one embodiment, the catalyst particles are monodisperse, having a coefficient of variation or relative standard deviation, expressed as a percentage and defined as the ratio of the particle diameter standard deviation (σ) to the particle diameter mean (μ), multiplied by 100%, of less than 25%, preferably less than 10%, preferably less than 8%, preferably less than 6%, preferably less than 5%. In a preferred embodiment, the catalyst particles are monodisperse having a particle size distribution ranging from 80% of the average particle size (e.g. diameter) to 120% of the average particle size, preferably 85-115%, preferably 90-110% of the average particle size. In another embodiment, the catalyst particles are not monodisperse.

In one or more embodiments, the catalyst of the present disclosure in any of its embodiments has a BET surface area of 200-700 m²/g, preferably 250-600 m²/g, preferably 300-500 m²/g, preferably 350-450 m²/g. In one embodiment, when SBA-15 is used as the mesoporous silica support, the catalyst has a BET surface area of 200-500 m²/g, 220-400 m²/g, 240-300 m²/g, or about 270 m²/g. In another embodiment, when mesoporous silica foam is used as the mesoporous silica support, the catalyst has a BET surface area of 300-700 m²/g, 340-600 m²/g, 360-450 m²/g, or about 390 m²/g.

In one or more embodiments, the catalyst of the present disclosure in any of its embodiments has an average pore diameter of 2-30 nm, preferably 3-20 nm, preferably 4-18 nm, preferably 5-16 nm, preferably 8-12 nm. In one embodiment, when SBA-15 is used as the mesoporous silica support, the catalyst has an average pore diameter of 2-10 nm, 3-8 nm, 3.5-6 nm, or about 4 nm. In another embodiment, when mesoporous silica foam is used as the mesoporous silica support, the catalyst has an average pore diameter of 10-30 nm, 12-20 nm, 14-18 nm, or about 16 nm.

In one or more embodiments, the catalyst of the present disclosure in any of its embodiments has a pore volume of 0.1-3 cm³/g, preferably 0.2-2 cm³/g, preferably 0.3-1.5 cm³/g, preferably 0.5-1 cm³/g. In one embodiment, when SBA-15 is used as the mesoporous silica support, the catalyst has a pore volume of 0.1-1 cm³/g, 0.2-0.6 cm³/g, 0.25-0.4 cm³/g, or about 0.3 cm³/g. In another embodiment, when mesoporous silica foam is used as the mesoporous silica support, the catalyst has a pore volume of 1-3 cm³/g, 1.2-2 cm³/g, 1.4-1.8 cm³/g, or about 1.6 cm³/g.

The catalytic activity of many oxides in various processes is due to their Lewis and/or Bronsted acidities. Depressing surface acidity and metal-support interactions of the catalyst may enhance butadiene selectivity in oxidative dehydrogenation reactions and reducing coke (COₓ) formation. The catalyst acidity plays a role in metal-support interactions that affect VOₓ reducibility. The reducibility may impact catalyst activity and selectivity by providing O₂ for oxidation and high acidity not favoring selective oxidation. A number of techniques have been developed for the characterization of acid-base surface properties of catalysts.

The adsorption of volatile amines including, but not limited to, ammonia (NH₃), pyridine (C₅H₅N), n-butylamine (CH₃CH₂CH₂CH₂NH₂), quinoline (C₉H₇N) and the like is often used to determine the acid site concentration of solid catalysts. The amount of the base remaining on the surface after evacuation is considered chemisorbed and serves as a measure of the acid site concentration. The adsorbed base concentration as a function of evacuation temperature can give a site strength distribution. Similarly, the basic site concentration of solid catalysts may be investigated using CO₂ as the standard probe molecule.

Another means of determining the site strength distribution is calorimetry or the temperature-programmed desorption (TPD). Ammonia TPD (NH₃-TPD) and CO₂-TPD experiments are used to determine the total acidity and basicity of the catalyst, respectively. TPD can further give an idea about metal-support interactions by modeling NH₃ and CO₂ desorption kinetics and be used to determine the strength of acid and basic sites available on the catalyst surface.

In a preferred embodiment, the acidity of the catalyst is quantified by measuring the amount of NH₃ adsorbed on the catalyst. In general, a catalyst having a stronger acidity adsorbs a greater amount of NH₃. In one embodiment, the catalyst of the present disclosure in any of its embodiments has a total acidity in the range of 0.02-0.2 mmol of NH₃ per gram of catalyst, preferably 0.04-0.18 mmol of NH₃ per gram of catalyst, preferably 0.06-0.16 mmol of NH₃ per gram of catalyst, preferably 0.08-0.14 mmol of NH₃ per gram of catalyst, preferably 0.1-0.12 mmol of NH₃ per gram of catalyst when measured at a temperature of 90-450° C., 100-400° C., 200-350° C., or 250-300° C. In a preferred embodiment, the basicity of the catalyst is quantified by measuring the amount of CO₂ adsorbed on the catalyst. In general, a catalyst having a stronger basicity adsorbs a greater amount of CO₂. In one embodiment, the catalyst has a total basicity in the range of 0.02-0.18 mmol of CO₂ per gram of catalyst, preferably 0.04-0.15 mmol of CO₂ per gram of catalyst, preferably 0.06-0.12 mmol of CO₂ per gram of catalyst, preferably 0.08-0.1 mmol of CO₂ per gram of catalyst when measured at a temperature of 150-450° C., 170-400° C., or 200-300° C.

In one embodiment, the use of SBA-15 as the mesoporous silica support may decrease the total acidity of the catalyst, preferably by less than 0.035 mmol of NH₃ per gram of catalyst, preferably by less than 0.03 mmol of NH₃ per gram of catalyst, preferably by less than 0.02 mmol of NH₃ per gram of catalyst, preferably by less than 0.01 mmol of NH₃ per gram of catalyst when measured at a temperature of 90-450° C., 100-400° C., 200-350° C., or 250-300° C. relative to a substantially similar catalyst opting a different support (e.g. MCM-41, Cariact Q-6, Cariact Q-10, Cariact Q-30, and silica foam). In another embodiment, the use of SBA-15 as the mesoporous silica support may increase the total basicity of the catalyst, preferably by at least 0.065 mmol of CO₂ per gram of catalyst, preferably by at least 0.06 mmol of CO₂ per gram of catalyst, preferably by at least 0.05 mmol of CO₂ per gram of catalyst, preferably by at least 0.04 mmol of CO₂ per gram of catalyst, preferably by at least 0.03 mmol of CO₂ per gram of catalyst when measured at a temperature of 150-450° C., 170-400° C., or 200-300° C. relative to a substantially similar catalyst opting a different support (e.g. MCM-41, Cariact Q-6, Cariact Q-10, Cariact Q-30, and silica foam). Overall, the use of SBA-15 as the mesoporous silica support may decrease the ratio of acid/base sites, preferably by 90%, preferably by 75%, preferably by 50%, preferably by 35% relative a substantially similar catalyst using a different support (e.g. MCM-41, Cariact Q-6, Cariact Q-10, Cariact Q-30, and silica foam) (see Example 3, Table 4).

According to a second aspect, the present disclosure relates to a method of preparing the catalyst of the first aspect, in one or more of its embodiments. The method involves (i) mixing the mesoporous silica support with an aqueous solution comprising a nickel salt and a bismuth salt to form a mixture, (ii) drying the mixture to form a dried mass, and (iii) calcining the dried mass in air at a temperature of 300-700° C., thereby producing the catalyst.

Two main methods are typically used to prepare supported catalysts. In the impregnation method, the solid support or a suspension of the solid support is treated with a solution of a precatalyst (for instance a metal salt or metal coordination complex), and the resulting material is then activated under conditions that will convert the precatalyst to a more active state, such as metal oxides of the metal or the metal itself. Alternatively, supported catalysts can be prepared from a homogenous solution by co-precipitation. In terms of the present disclosure, it is envisaged that the catalyst may be formed by an impregnation method or a co-precipitation method, preferably by an impregnation method. The mesoporous silica support used herein is preferably thermally stable and withstands processes required for precatalyst activation. For example, precatalysts may be activated by exposure to a stream of air (oxygen) or hydrogen at high temperatures, additionally precatalysts may be further activated and/or reactivated by oxidation-reduction cycles, again at high temperatures.

In one step of the method, the mesoporous silica support is mixed with an aqueous solution comprising a nickel salt and a bismuth salt to form a mixture. Typically a main method of disposing bismuth oxide on a support material is wet impregnation conducted in a solution where the bismuth and nickel oxides precursors are soluble. In one embodiment, the impregnation method is performed by contacting the mesoporous silica support with a certain volume of solution containing the dissolved bismuth and nickel salts. In one embodiment, the mesoporous silica support may be initially dried and/or calcined to remove volatile impurities. The initial drying may be performed at a temperature of 200-400° C., 250-350° C., or 300-320° C. for a period of up to 6 hours, preferably up to 3 hours, or about 1 hour. The initial calcining may be performed at a temperature of 450-800° C., 500-700° C., or 550-650° C. for a period of up to 12 hours, preferably up to 6 hours, or about 2 hours.

In one embodiment, the bismuth salt may be a bismuth(III) salt. Exemplary bismuth salts include, but are not limited to, bismuth(III) nitrate, bismuth(III) sulfate, bismuth(III) acetate, bismuth(III) chloride, bismuth(III) bromide, bismuth(III) iodide, bismuth(III) phosphate, bismuth hydroxide, bismuth(III) citrate, bismuth(III) oxynitrate, bismuth(III) oxychloride, and the like. In one embodiment, more than one type of bismuth(III) salt may be used. Preferably, the bismuth salt is bismuth(III) nitrate. In one embodiment, the nickel salt may be a nickel(II) salt, though in an alternative embodiment, nickel having a different oxidation state, such as +3, may be used. Exemplary nickel salts include, but are not limited to, nickel(II) nitrate, nickel(II) sulfate, ammonium nickel(II) sulfate, nickel(II) acetate, nickel(II) chloride, nickel(II) bromide, nickel(II) iodide, nickel(II) perchlorate, and the like. In one embodiment, more than one type of nickel(II) salt may be used. Preferably, the nickel salt is nickel(II) nitrate. In a preferred embodiment, the solvent is a polar protic solvent. Exemplary polar protic solvents include, and are not limited to, water, methanol, ethanol, iso-propanol, and n-butanol. Preferably, the solvent is water. It is equally envisaged that the present method may be adapted to incorporate polar aprotic solvents such as acetone, tetrahydrofuran, ethyl acetate, dimethylformamide, acetonitrile, and dimethyl sulfoxide, as well as non-polar solvents such as pentane, hexane, cyclohexane, benzene, toluene, 1,4-dioxane, chloroform, diethyl ether, and dichloromethane, and mixtures thereof.

In a most preferred embodiment, the solution contains water as the solvent, bismuth(III) nitrate as the bismuth salt, and nickel(II) nitrate as the nickel salt. The aqueous solution has the nickel salt at a concentration of 20-80 mM, preferably 30-70 mM, preferably 35-60 mM, or about 40 mM. The aqueous solution has the bismuth salt at a concentration of 10-40 mM, preferably 12-30 mM, preferably 14-25 mM, preferably 16-20 mM, or about 18 mM. In a preferred embodiment, the weight ratio of bismuth to nickel atoms in the solution is in the range of 5:1 to 1:2, preferably 4:1 to 1:1, or about 3:2. In some embodiments, the weight ratio of bismuth atoms to the mesoporous silica support ranges from 1:2 to 1:6, preferably 1:3 to 1:5, or about 3:10. In some embodiments, the weight ratio of nickel atoms to the mesoporous silica support ranges from 1:2 to 1:10, preferably 1:3 to 1:8, preferably 1:4 to 1:6, or about 1:5. In one embodiment, a weight ratio of the mesoporous silica support to the nickel salt ranges from 1:3 to 3:1, preferably 1:2 to 2:1, or about 1:1. A weight ratio of the mesoporous silica support to the bismuth salt may range from 1:2 to 4:1, preferably 2:3 to 2:1, or about 4:3.

In a preferred embodiment, mixing the mesoporous silica support with an aqueous solution comprising a nickel salt and a bismuth salt is performed at a temperature of 10-50° C., preferably 20-40° C., or about 25° C. for a period of 4-24 hours, 8-20 hours, or 12-16 hours and optionally with stirring and/or ultrasonication to achieve a homogeneous mixture.

The method also involves the step of drying the mixture to form a dried mass. Preferably this step involves removing the solvent (e.g. water) from the mixture and facilitating deposition of nickel species on the mesoporous silica support. In one embodiment, this step involves heating the mixture to evaporate the solvent. For example, the mixture may be heated at 60-100° C., 70-90° C., or about 80° C. in order to remove the water. In certain embodiments, the mixture may be further heated at 100-150° C., 110-130° C., or about 120° C. for 1-6 hours, 2-4 hours, or about 3 hours to form a dried mass. In other embodiments, the mixture may be subjected to a vacuum, or a rotary evaporator. In another embodiment, the mixture may be heated in an oven, or left at room temperature.

The dried mass may be calcined in air within a furnace or oven at a temperature of 300-700° C., 350-600° C., or 400-500° C., though in some embodiments, the dried mass may be heated at a temperature of lower than 300° C. or higher than 700° C. In some embodiments, the dried mass may not be heated in air, but oxygen-enriched air, an inert gas, or a vacuum. Preferably the dried mass is placed in an oven at room temperature or 20-50° C., and then the temperature is increased to a first target calcining temperature of 300-450° C., 325-400° C., or about 350° C. at a rate of 5-15° C./min, preferably 8-12° C./min, or about 10° C./min. The dried mass may be maintained at the first target calcining temperature for 0.1-3 hours, 0.5-2 hours, or about 1 hour. Preferably the temperature of the oven is further increased to a second target calcining temperature of 450-700° C., 500-650° C., or about 590° C. at a rate of 10-20° C./min, 12-18° C./min, or about 15° C./min. The dried mass is maintained at the second target calcining temperature for 0.5-6 hours, 1-4 hours, or about 2 hours. Calcining the dried mass produces the catalyst.

In another embodiment, it is equally envisaged that the method may be adapted to other means of dispersing and impregnating the bismuth and nickel oxides on the mesoporous silica support. Exemplary other means include, but are not limited to, isomorphous substitution, enforced impregnation, vapor-fed flame synthesis, flame spray pyrolysis, sputter deposition, atomic layer deposition, and chemical vapor deposition.

According to a third aspect, the present disclosure relates to a process of oxidatively dehydrogenating n-butane to form butadiene and butenes, the process involving flowing a feed mixture comprising n-butane and an oxidant through a reactor loaded with the catalyst of the present disclosure, in one or more of its embodiments, thereby forming butadiene and butenes.

As defined herein, a butene refers to 1-butene, (Z)-but-2-ene, (E)-but-2-ene, or mixtures thereof “Butenes” used herein refers to an aggregate of all butenes generated during the process of oxidative dehydrogenation.

As used herein, dehydrogenation refers to a chemical reaction that involves the removal of hydrogen from a molecule. It is the reverse process of hydrogenation. The dehydrogenation reaction may be conducted on both industrial and laboratory scales. Essentially, dehydrogenation converts saturated compounds to unsaturated compounds and hydrogen. Dehydrogenation processes are used extensively in fine chemicals, oleochemicals, petrochemicals and detergents industries. The catalytic dehydrogenation of alkanes is more selective to particular degrees of dehydrogenation but the reaction characteristics pose inherent difficulties and impose certain technical constraints. For example, thermal dehydrogenation is strongly endothermic and often requires operation at both high temperature and high alkane partial pressure. The oxidative dehydrogenation (ODH) of an alkane, which couples the endothermic dehydrogenation of the alkane with the strongly exothermic oxidation of hydrogen avoids the need for excess internal heat input and consumes hydrogen.

The performance of the oxidative dehydrogenation can be modulated by adjusting conditions including, but not limited to, ratio of oxidant/reactant, temperature, pressure, catalyst loading, and/or reaction time.

In one or more embodiments, the oxidant and n-butane are present in the feed mixture at a molar ratio in a range of 0.1:1 to 8:1, preferably 0.5:1 to 6:1, preferably 1:1 to 4:1, or about 2:1. In a preferred embodiment, the oxidant is O₂. Other oxidants such as air may be useful for the present disclosure. In one or more embodiments, the feed mixture further comprises an inert gas such as N₂, Ar, and/or He. In a preferred embodiment, a N₂ stream is mixed with the oxidant (e.g. O₂) and n-butane before the oxidative dehydrogenation process. In a preferred embodiment, the oxidative dehydrogenation of n-butane to butadiene and butenes is carried out by flowing the feed mixture at a flow rate of 5-200 mL/min, 10-100 mL/min, 20-50 mL/min, or 25-35 mL/min. In a preferred embodiment, the feed mixture is flowed at a temperature in the range of 300-700° C., preferably 350-650° C., preferably 400-600° C., preferably 410-550° C., preferably 420-525° C., preferably 430-500° C., or about 450° C., and preferably at a pressure of 35-350 kPa, 50-300 kPa, 75-200 kPa, or about 100 kPa. In a preferred embodiment, the catalyst loading or amount of catalyst present in the oxidative dehydrogenation reaction is in the range of 0.01-1.0 g of catalyst per mL of n-butane feed injected, preferably 0.05-0.8 g/mL, preferably 0.1-0.6 g/mL, preferably 0.2-0.4 g of catalyst per mL of n-butane feed injected. The conditions may vary from these ranges and still provide acceptable conditions for performing the oxidative dehydrogenation of n-butane to butadiene and butenes utilizing the catalyst of the present disclosure.

Oxidative dehydrogenation catalysts are evaluated for their percent conversion of the alkane as well as their selectivity to a product (i.e. the corresponding alkene, diene, or COₓ such as CO and/or CO₂). The selectivity of the catalyst to dehydrogenation products DH (consisting essentially of 1-butene, 2-butenes and 1,3-butadiene (BD)), oxygenate and cracked products OC (carboxylic acids, ethylene, propylene, methane, and CO₂) and partial oxidation products PO (CO and H₂) are summarized in Example 4, Tables 6 and 7. The definitions used in calculating the conversion and selectivity are represented for the method of the present disclosure using the oxidative dehydrogenation catalyst are represented in formula (I) and formula (II) respectively.

$$\begin{matrix}Conversion of alkane = \frac{Moles of alkane converted}{Moles of alkane fed} \times 100 \% & ( I ) \\ Selectivity to product = \frac{Moles of product}{\begin{matrix}Moles of alkane reacted - \\ Moles of product \end{matrix} } \times 100 \% & ( II )\end{matrix}$$

Compared to conventional silica, mesoporous silica species have ordered pore structures and large surface areas, which may enhance dispersion of active catalysts thereby generating more active sites. Also, mesoporous silica supports have well-defined pore sizes and thick pore walls. The use of the mesoporous silica support (e.g. SBA-15 (catalyst D), mesoporous silica foam (catalyst F)) makes the catalyst reach a greater overall catalytic activity than a catalyst that is formed with a conventional silica support (e.g. Cariact Q-6 (catalyst A), Cariact Q-10 (catalyst B), Cariact Q-30 (catalyst C)) or with a different mesoporous silica support (e.g. MCM-41 (catalyst E)) (see Example 4 by comparing Tables 6 and 7).

In one or more embodiments, the process of the present disclosure has an oxidative dehydrogenation n-butane conversion rate of up to 45%, preferably up to 40%, preferably up to 35%, preferably up to 30%, such as for example 5-40%, preferably 10-36%, preferably 12-32%, more preferably 15-29% and at least 6%, preferably at least 16%, preferably at least 22%, preferably at least 26%, preferably at least 30%. In one or more embodiments, the process using the catalyst of the present disclosure may lead to an n-butane conversion rate 20-65% greater, preferably 30-60% greater, more preferably 40-50% greater than a substantially identical catalyst not formed with the mesoporous silica support such as SBA-15 and/or mesoporous silica foam. Here, the substantially identical catalyst not formed with the mesoporous silica support such as SBA-15 and/or mesoporous silica foam may refer to a catalyst having bismuth and nickel oxides each present in relative weight percentages substantially similar to those in the currently disclosed catalyst, which are impregnated on a conventional silica support (e.g. Cariact Q-6, Cariact Q-10, Cariact Q-30) or on a different mesoporous silica support (e.g. MCM-41). In one embodiment, the process using the catalyst of the present disclosure may lead to an n-butane conversion rate 40-70% greater, preferably 45-60% greater, more preferably 50-55% greater than a substantially identical catalyst formed with a conventional silica support (e.g. Cariact Q-6, Cariact Q-10, Cariact Q-30). In another embodiment, the process using the catalyst of the present disclosure may lead to an n-butane conversion rate 30-60% greater, preferably 35-55% greater, more preferably 40-50% greater than a substantially identical catalyst formed with a different mesoporous silica support (e.g. MCM-41).

In one or more embodiments, the process of the present disclosure has a butadiene yield of up to 40 wt %, preferably up to 30 wt %, preferably up to 20 wt %, preferably up to 15 wt %, preferably up to 10 wt % relative to a weight of n-butane in the feed mixture, such as for example 5-35 wt %, preferably 8-25 wt %, more preferably 13-20 wt % relative to a weight of n-butane in the feed mixture, and at least 6 wt %, preferably at least 9 wt %, more preferably at least 14 wt % relative to a weight of n-butane in the feed mixture. In one embodiment, the process using the catalyst of the present disclosure may lead to a butadiene yield 50-90% greater, preferably 55-80% greater, more preferably 60-70% greater than a substantially identical catalyst formed with a conventional silica support (e.g. Cariact Q-6, Cariact Q-10, Cariact Q-30). In another embodiment, the process using the catalyst of the present disclosure may lead to a butadiene yield 40-80% greater, preferably 45-70% greater, more preferably 50-60% greater than a substantially identical catalyst formed with a different mesoporous silica support (e.g. MCM-41).

In one or more embodiments, the process of the present disclosure has a molar ratio of butadiene to butenes (e.g. 1-butene and 2-butenes) in a range of about 1:1 to about 4:1, preferably about 1.2:1 to about 3:1, preferably about 1.5:1 to about 2:1, or about 1.7:1. In one embodiment, the process of the present disclosure has an oxidative dehydrogenation butadiene selectivity of at least 60%, preferably at least 65%, preferably at least 70%, preferably at least 75%, preferably at least 80%, preferably at least 85%, preferably at least 90%, preferably at least 95% such as for example 60-90%, preferably 61-85%, preferably 62-75%, more preferably 63-70%. In one embodiment, the process using the catalyst of the present disclosure may lead to a butadiene selectivity 30-70% greater, preferably 40-60% greater, more preferably 45-50% greater than a substantially identical catalyst formed with a conventional silica support (e.g. Cariact Q-6, Cariact Q-10, Cariact Q-30). In another embodiment, the process using the catalyst of the present disclosure may lead to a butadiene selectivity 10-30% greater, preferably 15-25% greater, more preferably 18-20% greater than a substantially identical catalyst formed with a different mesoporous silica support (e.g. MCM-41). High butadiene selectivity demonstrated by the catalyst having SBA-15 as the mesoporous silica support may be related to its selective dehydrogenation ability shown in the small amount of 1-butene desorbed into the gaseous phase during the 2^{nd} step dehydrogenation.

In a preferred embodiment, the reactor is a fixed bed reactor. As used herein, a fixed bed reactor is a type of reaction device that contains catalyst, usually in pallet form, packed in one or more static beds. In this type of reactor, a feed stream is passed through static beds where reactions occur as the feed stream contacts the catalyst. In a fixed-bed reactor, the static beds are usually held in place and do not move with respect to the reactor. It is equally envisaged that the process of the present disclosure may be adapted to be performed in a fluidized bed reactor. In a fluidized bed reactor, a fluid (gas or liquid) is passed through a granular solid material (usually a catalyst, preferably spherically shaped) at high enough velocities to suspend the solid and cause it to behave as though it were a fluid.

The general nature of the alkane substrate (n-butane) is not viewed as particularly limiting to the oxidative dehydrogenation described herein. It is equally envisaged that the present disclosure may be adapted to oxidatively dehydrogenating other alkanes in addition to, or in lieu of n-butane to form corresponding alkenes. As used herein, “alkane” unless otherwise specified refers to both branched and straight chain saturated aliphatic primary, secondary, and/or tertiary hydrocarbons of typically C₁ to C₁₀. Other exemplary alkanes include, but are not limited to, straight or branched alkanes of C₁ to Cᵢ₀ such as ethane (C₂H₆), propane (C₃H₈), and isobutane, and the corresponding alkene such as ethylene, propylene, and isobutylene (2-methylpropene). Cycloalkanes, which are optionally substituted alicyclic hydrocarbons of typically C₃ to C₁₆, and specifically includes, but is not limited to, cyclopropane, cyclobutane, cyclopentane, cyclohexane, and cycloheptane, may also be oxidatively dehydrogenated using the catalyst of the present disclosure. In certain embodiments, the alkane may be sourced from other industrial processes such as those used in the petrochemical industry. Feedstocks generated from petroleum including, but not limited to, liquefied petroleum gas (LPG, or liquid petroleum gas), ethane, propane, butane, naphtha, pet naphtha, pygas, light pygas, and gas oil may serve as substrates for the method of oxidatively dehydrogenating an alkane described herein.

The examples below are intended to further illustrate protocols for preparing and characterizing the catalyst of the present disclosure. Further, they are intended to illustrate analyzing the properties and performance of these catalysts. They are not intended to limit the scope of the claims.

### Example 1

### Synthesis of Catalyst Support

Conventional silica supports having different pore diameters (Cariact Q-6, Cariact Q-10, and Cariact Q-30) were purchased from Fuji Silysia chemicals Ltd.

The MCM-41 silica support was synthesized following the procedure of Beck et al. [J. S. Beck, J. C. Vartuli, W. J. Roth, M. E. Leonowicz, C. T. Kresge, K. D. Schmitt, C. T.-U. Chu, D. H. Olsen, E. W. Sheppard, S. B. McCullen, J. B. Higgins, J. L. Schlenker, J. Am. Chem. Soc. 114, 10834 (1992), incorporated herein by reference in its entirety].

The SBA-15 silica support was synthesized using a tri-block copolymer, poly(ethylene glycol)-block-poly(propylene glycol)-block-poly(ethylene glycol), as a structure directing agent. In a typical synthetic procedure, 4 g of Pluronic® P123 was added to 30 ml of water. After stirring for a few hours, a clear solution was obtained. About 70 g of 0.28 M hydrochloric acid was added to it and the solution was stirred for another 2 h. Then, 9 g of tetraethyl orthosilicate (TEOS) was added and the resulting mixture was stirred for 24 h at 40° C., and eventually heated at 100° C. for 48 h. A solid product was recovered by filtration, washed with water for several times and dried overnight at 100° C. Finally, the product was calcined at 550° C. for 6 h to remove the template.

The mesoporous silica foam was synthesized using the following method. In a typical synthetic procedure, 3.0 g of a neutral triblock co-polymer surfactant, Pluronic® P123, was dissolved in a mixture of 3.0 g of acetic acid, 52 g of deionized water, and 0.3 g of ammonium fluoride at 40° C. After stirring for 2 h, a solution of sodium silicate (2.35 g) in water (40 g) was added and the resultant mixture was reacted under vigorous stirring for 5 min. Then, the mixture was kept under static condition for 24 h at 40° C. followed by aging at 70° C. overnight. The solid products were washed with deionized water, collected by filtration, and air dried. The obtained solid was then calcined at 560° C. for 6 h to remove the template.

### Example 2

### Preparation of Catalysts

The catalysts were prepared by a co-impregnation method of equilibrium adsorption with enforced deposition. In a typical synthesis, nickel nitrate hexahydrate Ni(NO₃)₂.6H₂O (99%, Fisher Scientific) was used as the nickel source, while bismuth nitrate pentahydrate Bi(NO₃)₃.5H₂O (98%, Fluka) was used as the bismuth source. To prepare a 20 wt % Ni-30 wt % Bi catalyst, 0.99 g of Ni(NO₃)₂.6H₂O was added to 80 mL of distilled water. After complete dissolution, 0.70 g of Bi(NO₃)₃.5H₂O was added and stirred. Then 1.0 g of dried support was added for impregnation and left overnight for aging to achieve equilibrium Bi species adsorption. Then the sample mixture was dried by evaporation at 80° C. for enforced Ni species deposition. The solid was further dried at 120° C. for 3 h, calcined at 350° C. (10° C./min) for 1 h. The solid was kept at a temperature, which was eventually raised to 590° C. (15° C./min), for 2 h. The obtained catalysts were each denoted as shown in Table 1.

TABLE 1
List of catalyst codes used for oxidative
dehydrogenation of n-butane to butadiene

| Catalyst Code | Description |
|---|---|
| A | 20 wt % Ni and 30 wt % Bi on Cariact Q-6 |
| B | 20 wt % Ni and 30 wt % Bi on Cariact Q-10 |
| C | 20 wt % Ni and 30 wt % Bi on Cariact Q-30 |
| D | 20 wt % Ni and 30 wt % Bi on Si-SBA-15 |
| E | 20 wt % Ni and 30 wt % Bi on Si-MCM-41 |
| F | 20 wt % Ni and 30 wt % Bi on silica foam |

### Example 3

### Catalyst Characterization

The physicochemical characteristics such as surface area and pore structure were analyzed by using nitrogen adsorption-desorption isotherm (Micromeritics ASAP 2020 instrument, Norcross, Ga.). The pore surface area, pore volume, and pore diameter were measured using BJH adsorption method. X-ray diffraction (XRD) of calcined samples were analyzed from (2 theta) range of 5° to 90° using Rigaku Miniflex II desktop X-ray diffractometer while using Cu Kα radiation (wavelength λ=1.5406λ) and 30 mA and 40 kV as operating parameters, a step size of 0.02° and a speed of 0.5°/min. The acid-base properties were analyzed using temperature programmed desorption (TPD) using the BEL-CAT-A-200 chemisorption instrument.

The redox character and acid-base property were analyzed using temperature programmed reduction (TPR) and temperature programmed desorption (TPD) using BEL-CAT-A-200 chemisorption instrument as reported earlier [B. R. Jermy, B. P. Ajayi, B. A. Abussaud, S. Asaoka, S. Al-Khattaf, J. Mol. Catal. A Chem. 400 (2015) 121, incorporated herein by reference in its entirety]. The redox property measurement was done using a gas mixture of Ar/H₂ (95/5 vol %) having a total flow rate of 50 cm³/min. 0.1 g of the calcined catalyst was preheated for 3 h at 300° C. in inert He after which it is cooled to room temperature. It was then heated at the rate of 20° C./min up to 900° C. H₂ intake was recorded with a TCD and CuO was used as a reference for calibrating the consumption of H₂.

### X-Ray Diffraction

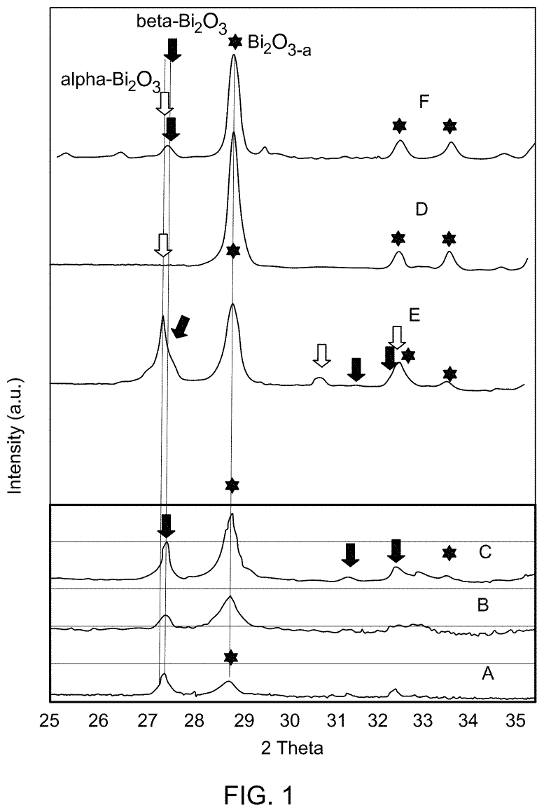

The X-ray diffraction (XRD) patterns related to Bi species for catalysts A to G are shown in FIG. 1. All SiO₂ supported catalysts contain NiO dispersed on BiOₓ, which are present on the various supports in a hierarchical cohabitation. The XRD patterns were measured from diffraction angle of 2θ=5 to 90°. Then, 2θ=25 to 35° was chosen to precisely examine the phases of BiOₓ in different catalysts. For the conventional SiO₂ supported catalysts (A-C), the main peaks are attributed to beta-Bi₂O₃ and Bi₂O₃₋ₐ at 2θ=27.38° and 29.0°, respectively. The intensity of these peaks increases as the pore size of the silica support increases. The micro and mesoporous silica supported catalysts showed intensities higher than the conventional SiO₂ supported catalysts, which indicates high crystallinity of the former resulted from their highly ordered structure and well-defined pores. Catalyst E (MCM-41 supported) showed two phases of BiOₓ at an approximate ratio of 40-60% between beta-Bi₂O₃ and Bi₂O₃₋ₐ. Highly dispersed NiO on this phase is selective for dehydrogenation of n-butane to butadiene. Catalyst F (mesoporous silica foam supported) also showed two BiOₓ phases with the dominant phase to be Bi₂O₃₋ₐ at 2θ=29.0°. Catalyst D (mesoporous SBA-15 supported) showed a pure and highly crystalline phase of Bi₂O₃₋ₐ. Using the XRD pattern of the pure and highly crystalline phase of BiOₓ and the phase assignment of Bi₆O₇ reported in [G. Tanimu, S. Asaoka, S. Al-Khattaf, Mol. Catal. 438 (2017) 245-255, incorporated herein by reference in its entirety], the phase was concluded to be Bi₂O₃₋ₐ (a=0.2-0.4). Though the XRD pattern agrees with previously assigned phase Bi₆O₇ (Bi₂O_{2.33}) in three major peaks according to the JCPDS Powder Diffraction File No. 27-0051, some minor but fingerprint-like peaks at 2 theta=5, 10 and 30.6° of (002), (004), and (0012), respectively could not be observed. Furthermore, the pattern did not agree with Bi₂O_{2.75} and Bi₂O_{2.5} in the data file. However, the positions of main peaks are between main peak positions at 31.92 and 27.93° corresponding to Bi₂O_{2.75} and Bi₂O_{2.5}, respectively. These studies lead to a conclusion that the phase is Bi₂O₃₋ₐ (a=0.2-0.4) phase. The peak intensity of the Bi₂O₃₋ₐ phase among the three mesoporous silica supported catalysts reflects their selectivity to butadiene.

The highly ordered mesoporous SBA-15 support was further loaded NiO and BiOx, separately and the XRD patterns were compared with that of the Ni—Bi—O/SBA-15 catalyst as shown in FIG. 3. For the NiO/SBA-15, peaks at 2θ=37 and 43° were observed corresponding to the NiO phase. The crystal size of the NiO was found to be 10.1 nm as determined from the Scherrer's equation. And for the case of BiOₓ/SBA-15, peaks of highly crystalline beta-Bi₂O₃ and Bi₂O₃₋ₐ were observed. It clearly indicates the formation of the pure and highly crystalline Bi₂O₃₋ₐ phase in SBA-15 supported catalyst is not only due to mesoporous SBA-15 but also accelerated by Ni species co-impregnation. As shown in FIG. 3, comparing Ni oxides catalyst alone with the binary catalyst, the dispersion of the NiO oxides was increased while the intensity of the peaks corresponding to the oxide was reduced. Only 30% of NiO with a crystal size of 20.9 nm (calculated using Scherrer's equation) was detected for catalyst D, indicating that 70% of NiO is highly dispersed on the Bi₂O₃₋ₐ surface. On the contrary, in the case of Bi amount reduction, 80% of NiO with a large crystal size of 20.9 nm was detected, indicating that only 20% is highly dispersed on the Bi₂O₃₋ₐ (a=0.2-0.4) surface.

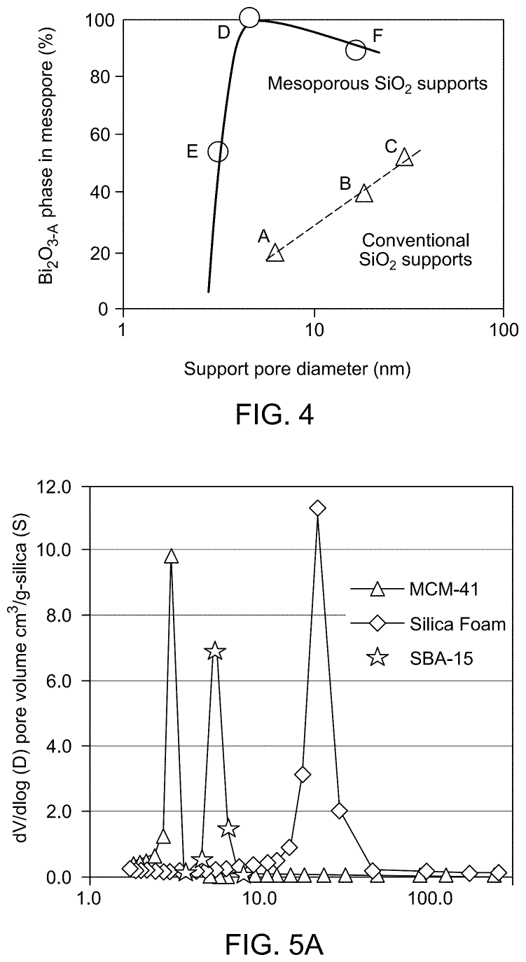

The concentration of Bi₂O₃₋ₐ phase on the support pore diameter is shown in FIG. 4. For the conventional silica supported catalyst, the concentration of the phase increases with increasing support pore diameter. This is a clear indication that, even within the amorphous phase, the crystallinity (silica arrangement like mesoporous silica) increases as the average pore diameter of the support increases. For the mesoporous structured silica supported catalysts, the Bi₂O₃₋ₐ phase concentration increases with increasing pore diameter up to 4.5 nm. For catalyst D, Bi₂O₃₋ₐ phase concentration was about 99.5%, whereas the catalyst F (with the highest average pore diameter) showed a decrease in Bi₂O₃₋ₐ phase concentration due to the presence of beta-Bi₂O₃ phase as observed in XRD.

### Surface Area and Pore Structure

The physicochemical properties of the catalysts were analyzed using various analytical techniques with the aim to investigate the nature of active sites and their varying degree of interactions and dispersions on different microporous and mesoporous silica support species. The specific surface area, pore surface area, pore volume, and the average pore diameters of the various catalysts are presented with corresponding silica supports in Table 2. All catalysts showed a reduced surface area compared to the one of their silica support. The pore structure (pore surface area and pore volume) of the catalysts followed a similar trend. But their values adjusted to support weight base showed either increased values or values decreased minimally. It is found that a high loading of active metal species unusually led to dispersion without causing pore blockages. Among the conventional silica supported catalysts, Catalyst A showed a surface area almost twice as that of Catalyst B and five times as that of Catalyst C. Their pore volumes, on the contrary, showed a different trend as catalyst B had the largest pore volume. Mesoporous silica supports usually have higher surface area and porosity than the conventional amorphous silica supports. However, the surface area and porosity of Catalyst D are clearly inferior to those of conventional amorphous silica supports. Catalyst D catalyst showed the best performance among other mesoporous silica materials as well as other conventional silica materials due to highly crystalline Bi₂O₃₋ₐ peak (contained 99.5% of the Bi₂O₃₋ₐ phase) as evident from XRD study.

TABLE 2
Physical properties of catalysts and supports.

| Catalyst: | | | | | | | Average |
|---|---|---|---|---|---|---|---|
| 20 wt % Ni— | BET surface area | | Pore surface area | | Pore volume | | pore |
| 30 wt % Bi—O/support | [m²/g- | [m²/g- | [m²/g- | [m²/g- | [cm³/g- | [cm³/g- | diameter |
| (Support) | catalyst]^{a} | support]^{b} | catalyst]^{c} | support]^{d} | catalyst]^{e} | support]^{f} | [nm]^{g} |
| A | 327 | 519 | 403 | 640 | 0.62 | 0.98 | 6.2 |
| (Cariact Q6) | | (426) | | (526) | | (0.83) | (6.3) |
| B | 158 | 251 | 171 | 272 | 0.71 | 1.13 | 16.8 |
| (Cariact Q10) | | (242) | | (263) | | (1.22) | (18.6) |
| C | 62 | 98 | 59 | 94 | 0.52 | 0.82 | 35.3 |
| (Cariact Q30) | | (166) | | (171) | | (1.28) | (29.9) |
| D | 269 | 427 | 309 | 491 | 0.33 | 0.52 | 4.2 |
| (Si-SBA-15) | | (657) | | (1080) | | (1.08) | (4.1) |
| E | 768 | 1220 | 761 | 1210 | 0.53 | 0.84 | 2.8 |
| (Si-MCM-41) | | (914) | | (1120) | | (0.86) | (3.1) |
| F | 388 | 616 | 397 | 630 | 1.55 | 2.46 | 15.6 |
| (Silica foam) | | (540) | | (554) | | (2.27) | (16.4) |

^{a}BET surface area,
^{c,e,g}Surface area, pore volume and average pore diameter measured using BJH isotherm,
^{b,d,f}Surface area and pore volume calculated to support weight base by using the equation: SA or PV × [Σ(MOₓ/M) + 100]/100, where M = metal wt %; MOₓ= metal oxide wt %; SA = surface area; PV = pore volume.

Though the mesoporous silica supports mostly have higher surface area and porosity than the conventional amorphous silica supports. The surface area of the catalysts adjusted to the support content in the catalysts is in more than half of the catalysts slightly higher than surface area of the pure support. Such observation is reported in our previous studies [B. R. Jermy, B. P. Ajayi, B. A. Abussaud, S. Asaoka, S. Al-Khattaf, J. Mol. Catal. A Chem. 400 (2015) 121; B. R. Jermy, S. Asaoka, S. Al-Khattaf, Catal. Sci. Technol. 5 (2015) 4622; G. Tanimu, B. R. Jermy, S. Asaoka, S. Al-Khattaf, J. Ind. Eng. Chem. 45 (2017) 111; and G. Tanimu, S. Asaoka, S. Al-Khattaf, Mol. Catal. 438 (2017) 245, each incorporated herein by reference in their entirety]. This finding was considered due to dispersion of Ni—Bi—O nano-sized particles.

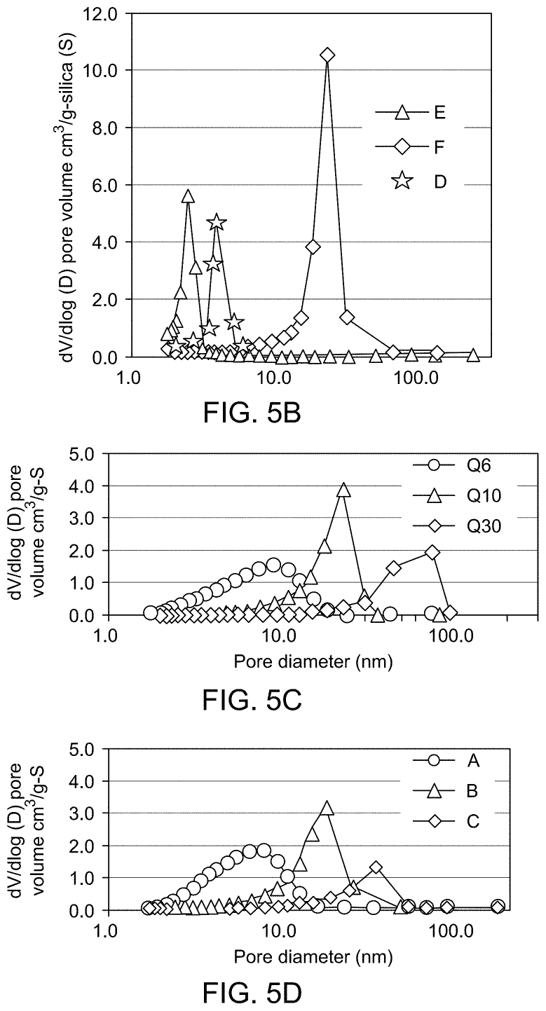

The pore size distributions of the mesoporous silica are narrow compared to the conventional silica, as shown in FIGS. 5A-5D. It also shows sharp (narrow) pore size distributions of the mesoporous silica catalysts, which are originated from the corresponding support without large shift in respective peak position.

### Temperature Programmed Reduction (TPR)

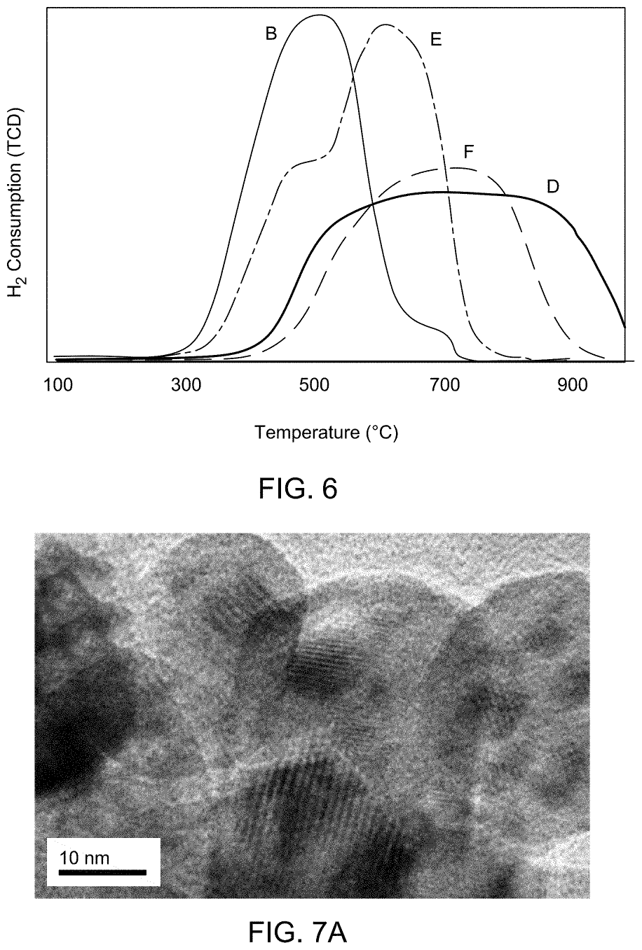

The extent of active species reducibility plays an important role in the activity and selectivity of catalysts, especially in oxidative dehydrogenation reactions. This property was measured using temperature programmed reduction (TPR). The performance of a catalyst is enhanced by an accelerated redox cycle resulting from increased active species reducibility. H₂-TPR profile of the mesoporous silica supported catalysts is shown in FIG. 6 in comparison with the conventional silica supported catalyst B. The TPR maximum temperature depends on the sample mass and the speed of the reduction reaction, hence same condition in terms of sample mass, the flow rate of H₂, and temperature programming was adopted.

The TPR profile of catalyst B showed a high-intensity reduction peak at 500° C. and a small reduction peak extending up to 700° C. from 625° C. The easy reduction of Ni—Bi—O metal oxide species at 500° C. shows the presence of larger particles which are not firmly interacting with the support. This change of reducibility over SiO₂ is related to the different states of NiO and Bi₂O₃ coordination between each other [G. Tanimu, S. Asaoka, S. Al-Khattaf, Mol. Catal. 438 (2017) 245, incorporated herein by reference in its entirety]. TPR profiles were categorized into three parts: I) easily reducible (350-550° C.), II) moderately reducible (550-650° C.) and III) difficult to reduce (650-850° C.) with the total as shown in Table 3. For the case of catalyst (MCM-41), a medium reduction peak at the easily reducible region and high intensity peak at around 700° C. were observed. It is also similar to the case of F (silica foam), even though the peak at the easily reducible region decreases and that of the region with difficulty increases in reducibility. The difficulty is related to the state of the smaller particles of highly dispersed active species strongly interacting with the mesoporous support species.

In the case of D (SBA-15), TPR profile showed a broad peak extending from 500-1000° C. The broadness of the reduction peak is related to the strong dispersion/interaction of the active species of NiO and BiOx with the supports.

TABLE 3
H₂ consumption in TPR of 20 wt % Ni-30
wt % Bi—O supported catalysts

| | TPR: Hi consumption [m mol/g] (T_{M}[° C.]) | | | |
|---|---|---|---|---|
| | I (350- | II (550- | III (650- | |
| Catalyst (Support) | 550° C.) | 650° C.) | 850° C.) | total |
| B (Cariact Q-10) | 3.74 | 1.12 | 0.13 | 4.99 |
| D (MCM-41) | 1.29 | 2.97 | 0.68 | 4.95 |
| E (SBA-15) | 0.88 | 1.04 | 2.43 | 4.35 |
| F (SiO₂ foam) | 0.70 | 1.01 | 2.51 | 4.22 |

Temperature Programmed Desorption (NH₃ and CO₂)

The basicity and acidity of the catalysts were measured using CO₂— and NH₃-temperature programmed desorption technique (CO₂— and NH₃-TPD), respectively. The amount of NH₃ and CO₂ desorbed in mmol/g for catalysts having 20 wt % Ni-30 wt % Bi—O metal oxides over different conventional, micro and mesoporous silica support species are presented in Table 4. CO₂-TPD profiles of the catalysts are deconvoluted into three peaks centered at around 170° C., around 300° C., and around 400° C., which were named as I, II, and III assigned to weak base, moderate base, and strong base, respectively. Similarly, NH₃-TPD profiles were also deconvoluted into three peaks with ranges at 100-250° C., 250-400° C., and around 400° C. named as I, II, and III representing weak acid, moderate acid, and strong acid, respectively.

Our previous reports showed that moderate and strong bases are required for efficient abstraction of H from terminal methyl of n-butane and 1-butene, and a weak acid site is required for 1-butene intermediate adsorption [G. Tanimu, B. R. Jermy, S. Asaoka, S. Al-Khattaf, J. Ind. Eng. Chem. 45 (2017) 111; and G. Tanimu, S. Asaoka, S. Al-Khattaf, Mol. Catal. 438 (2017) 245, each incorporated herein by reference in their entirety]. All the conventional silica supported catalysts do not possess moderate or strong basic site, whereas mesoporous silica supported catalysts have both moderate and strong basic sites. Catalyst B has the highest total basic sites and weak acid sites among the conventional silica supported catalysts. All the mesoporous silica supported catalysts have both weak and moderate acid sites without strong acid sites. Catalyst D has the highest moderate basic sites, which plays an important role in butadiene selectivity.

TABLE 4
Temperature programmed desorption (CO_{2—} and NH₃_{_{—}}
TPD) of 20 wt % Ni-30 wt % Bi—O/support

| | CO₂-TPD | | | | NH₃-TPD | | | | |
|---|---|---|---|---|---|---|---|---|---|
| Catalyst | Base amount [m mol/g]*¹ | | | | Acid amount [m mol/g]*² | | | | Acid/ |
| (Support) | I | II | III | Total | I | II | III | Total | Base |
| A (Cariact Q-6) | 0.014 | 0.008 | — | 0.022 | 0.019 | 0.010 | 0.003 | 0.032 | 1.5 |
| B (Cariact Q-10) | 0.031 | 0.002 | — | 0.033 | 0.039 | 0.014 | 0.001 | 0.054 | 1.6 |
| C (Cariact Q-30) | 0.009 | 0.002 | — | 0.011 | 0.011 | 0.002 | — | 0.013 | 1.2 |
| D (SBA-15) | 0.056 | 0.014 | 0.004 | 0.074 | 0.044 | 0.021 | — | 0.065 | 0.9 |
| E (MCM-41) | 0.08 | 0.002 | 0.011 | 0.021 | 0.026 | 0.039 | — | 0.065 | 3.1 |
| F (Silica foam) | 0.026 | 0.006 | 0.011 | 0.043 | 0.053 | 0.041 | — | 0.094 | 2.2 |

### TEM Analysis

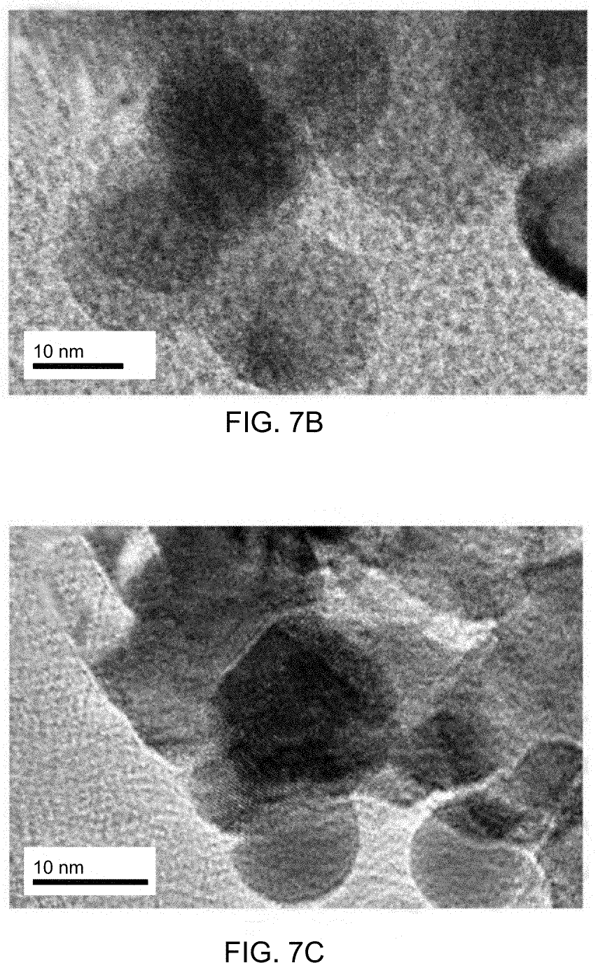

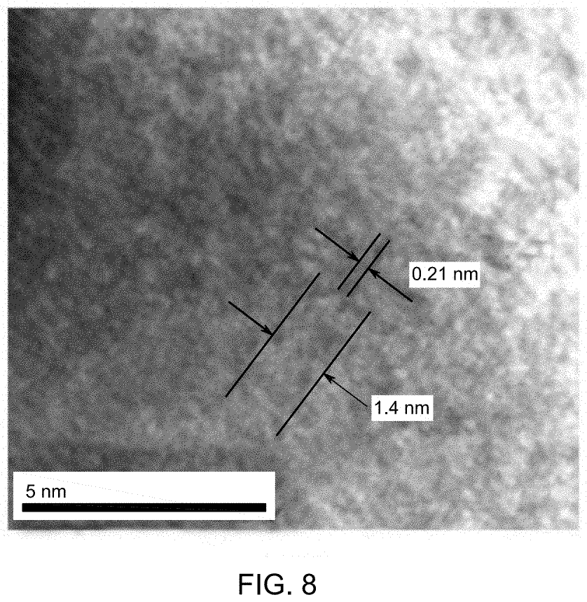

Transmission electron microscopy images (TEM) of the mesoporous supported catalysts having 20 wt % Ni and 30 wt % Bi are shown in FIGS. 7A-C. TEM images of conventional silica have been reported in our previous report [G. Tanimu, S. Asaoka, S. Al-Khattaf, Mol. Catal. 438 (2017) 245, incorporated herein by reference in its entirety]. The crystals of BiOₓ in the conventional silica were too small to be observed by TEM, probably due to a low crystallinity as evident from XRD study. The TEM images showed the dispersion of the NiO with the BiOₓ on various supports. The mesoporous support showed a similar pattern of active species dispersion. To precisely investigate the active species dispersion, high-resolution transmission electron microscopy (HRTEM) was carried out on the catalyst D to enable the determination of the Bi₂O₃₋ₐ crystal and NiO crystal. FIG. 8 shows the HRTEM image of the Catalyst D. The observed HRTEM image showed a lattice spacing of 0.21 nm and 1.4 nm corresponding to that of NiO and SBA-15, respectively. As the 1.4 nm lattice of SBA-15 reflects the orientation of silica chains, the lattices of NiO are also oriented in the same direction. A major part of NiO is not dispersed directly on SBA-15, but through Bi₂O₃₋ₐ phase in SBA-15.

This means the three oxide chains, (i) silica chain in SBA-15 mesopore, (ii) Bi—O chain of Bi₂O₃₋ₐ phase and (iii) Bi—O dispersed on Bi₂O₃₋ₐ phase have the same orientation. The TEM image of the SBA-15 supported catalyst also agreed with the previous literature report [C. J. Gommes, H. Friedrich, M. Wolters, P. E. de Jongh, K. P. de Jong, Chem. Mater. 21 (2009) 1311, incorporated herein by reference in its entirety].

### Example 4

### Catalytic Performance of Ni and Bi Loaded Catalysts

### Effect of Reaction Condition

The reaction temperature and O₂/n-C₄H₁₀ feed ratio were varied to test the performance of Catalyst D. The dependence of activity/selectivity on time-on-stream was observed only until one hour (second sampling) due to initial oxygen species on catalyst surface. The result obtained at 5 h time-on-stream is presented in Table 5.

TABLE 5
Comparison of catalytic performance over 20
wt % Ni-30 wt % Bi—O/SBA-15 catalyst (catalyst
D) at different reaction conditions

| | Reaction temperature [° C.] | | | | |
|---|---|---|---|---|---|
| | 400 | 400 | 400 | 450 | 500 |
| O₂/n—C₄H₁₀ molarratio | 1.0 | 2.0 | 4.0 | 2.0 | 2.0 |
| n-C₄H₁₀ conversion[%] | 21.5 | 26.5 | 25.4 | 28.9 | 37.7 |
| Selectivity*¹ [C %] | | | | | |
| DH | 91.6 | 87.6 | 80.1 | 75.2 | 49.6 |
| 2-C₄H₈ | 23.3 | 20.5 | 19.7 | 15.4 | 15.5 |
| 1-C₄H₈ | 18.8 | 19.1 | 18.9 | 12.3 | 4.3 |
| BD | 49.5 | 48.0 | 41.5 | 47.5 | 38.2 |
| OC | 6.7 | 9.3 | 11.3 | 19.7 | 43.3 |
| PO | 1.7 | 3.2 | 8.5 | 5.1 | 7.1 |
| (1-C₄H₈ +BD)*² | 68.3 | 67.1 | 60.4 | 59.8 | 42.5 |
| BD/(1-C₄H₈ + BD)%*³ | 72.5 | 71.5 | 68.7 | 79.4 | 89.8 |
| BD yield | 10.7 | 12.7 | 10.5 | 13.7 | 14.4 |

*¹DH: dehydrogenation, BD: butadiene, OC: Oxygenate and the cracked. PO: partial oxidation.
*²selectivity at 1^{st} step dehydrogenation,
*³selectivity at 2^{nd} step dehydrogenation

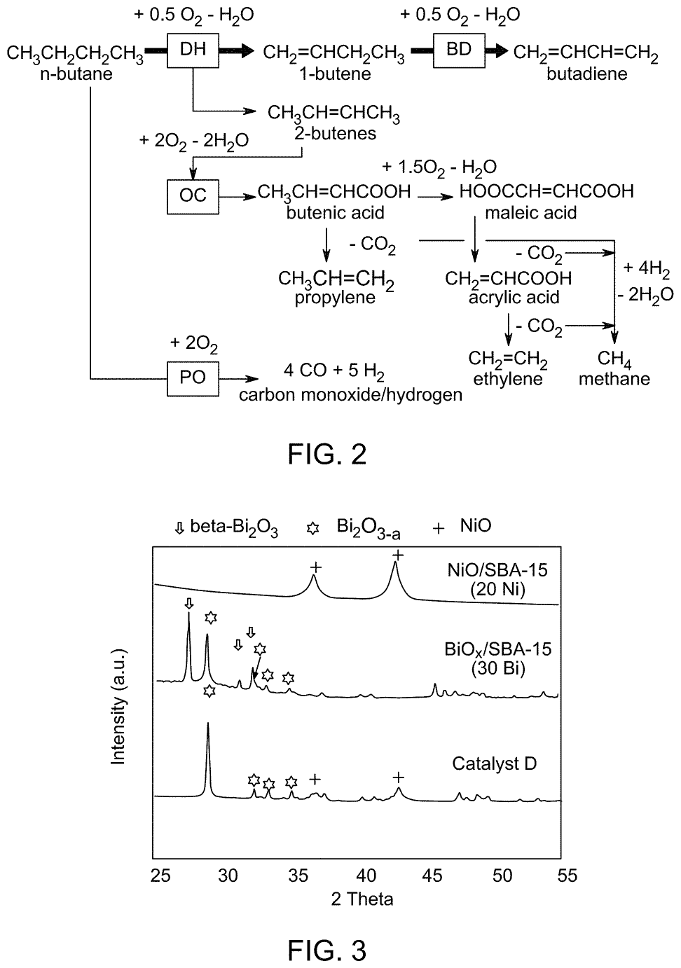

The result confirms the reaction pathway proposed in FIG. 2 that oxygenate and cracking products are obtained mainly from 2-butene after the 1st dehydrogenation step. With an increase in the reaction temperature, the conversion increased, while dehydrogenation selectivity decreased. BD selectivity did not change but OC production increased two folds with an increase in temperature. This is due to excess NiO species, which are more active at higher temperatures and favoring cracking reaction pathway. Upon increasing O₂/n-C₄H₁₀ feed ratio, the conversion increased up to 2.0 ratio, thereafter a slight decrease in conversion was observed. With an increase in O₂/n-C₄H₁₀ feed ratio, the dehydrogenation selectivity and butadiene selectivity decreased, and OC and PO selectivity increased due to excess oxygen supply which facilitated cracking and oxidation reactions. The decrease in conversion with increasing O₂/n-C₄H₁₀ feed ratio from 2.0 to 4.0 suggests oxygen species saturation of major and selective dehydrogenation sites. The oxygenate byproducts suppress main reaction, resulting a decrease in conversion.

The oxidative dehydrogenation was carried out in an automated fixed bed reactor purchased from BELCAT, Japan. 0.3 g of catalyst was loaded into the reactor and calcined under air atmosphere. After calcination, the reaction was initiated under a nitrogen atmosphere. The contact time of n-butane feed was maintained at 0.42 h·g/mol. The total flow rate of reactants including n-butane, air, and nitrogen was maintained at 31.2 mL/min. The effect of different temperatures (400, 450 and 500° C.) and various oxygen to n-butane ratio (O₂/n-C₄H₁₀=1.0, 2.0 and 4.0 mol/mol) was tested. The products were analyzed using an online GC system (Agilent, 7890N). The hydrocarbons and oxygenates were analyzed using FID and a GC-Gas Pro capillary column (L: 60 m and ID: 0.32 mm). Detection of different gases such as N₂, O₂, CO, CO₂, and H₂ was performed using TCD and Shin Carbon 80/100 mesh SS column (He carrier), and MSSA 60/80 mesh SS column (Ar carrier). The identities of products were confirmed by comparing with standard samples. The n-butane conversion and product selectivity were measured using the mass balance of carbon.

### Effect of Conventional Silica Support

The effect of different pore sizes of commercially available silica gel support on the catalytic performance of 20 wt % Ni-30 wt % Bi on SiO₂ support for the oxidative dehydrogenation of n-butane to butadiene was studied under standard conditions of 450° C. with O₂/n-C₄H₁₀=2.0. The results are presented in Table 6.

As shown in Table 6, while catalyst A had a slightly higher conversion among the catalysts, all catalysts had similar n-butane conversions. This indicated a similar extent of active species dispersed on the catalysts. For main reaction selectivity, catalyst B showed the highest dehydrogenation selectivity with the lowest oxygenates and cracked products selectivity. This observation supported a trade-off relationship between the main two reaction selectivity of the catalyst. Catalyst A produced the largest amount of 2-butenes as well as a high selectivity of cracked products. This is an indication that excess dispersion of Ni species may favor such reaction pathway. All catalysts demonstrated similar selectivity to 1-butene, which suggested that the 2^{nd} step dehydrogenation selectivity was similar. Butadiene selectivity was similar among medium and large porous materials supported catalysts, and slightly lower in the case of the small porous material supported catalyst. This indicates that a relatively large pore size is required for conventional silica support to effectively and efficiently disperse BiOₓ and NiO species. In general, the overall performance of the three catalysts supported by silica having different pore sizes are similar according to their 1^{st} and 2^{nd} step dehydrogenation selectivity and butadiene yields.

TABLE 6
Comparison of catalytic performance of conventional
SiO₂ gel with different pore sizes supported Ni—Bi—O catalysts.
Catalyst: 20 wt % Ni-30 wt % Bi—O/SiO₂ gel. Reaction condition: 450°
C., O₂/n-C₄H₁₀ = 2.0

| | Catalyst | | |
|---|---|---|---|
| | A | B | C |
| Support APD [nm] | 6.3 | 18.6 | 29.9 |
| n-C₄H₁₀ conversion [%] | 19.8 | 17.6 | 18.2 |
| (O₂ conversion) | (32) | (25) | (31) |

Selectivity*¹ [C %]

| | | | |
|---|---|---|---|
| DH | 74.3 | 79.1 | 75.9 |
| 2-C₄H₈ | 24.5 | 21.7 | 18.4 |
| 1-C₄H₈ | 22.3 | 25.5 | 25.1 |
| BD | 27.5 | 31.9 | 32.4 |
| OC | 22.7 | 17.5 | 21.5 |
| PO | 3.0 | 3.4 | 2.5 |
| (1-C₄H₈ + BD)*² | 49.8 | 57.4 | 57.5 |
| BD/(1-C₄H₈ + BD) %*³ | 55.2 | 55.6 | 56.4 |
| BD yield | 5.4 | 5.6 | 5.9 |

*¹DH: dehydrogenation, BD: butadiene, OC: oxygenate and the cracked, PO: partial oxidation.
*²selectivity at 1^{st} step dehydrogenation,
*³selectivity at 2^{nd} step dehydrogenation

### Effects of Mesoporous Silica Support

Three different mesoporous silica support species (MCM-41, SBA-15 and mesoporous SiO₂ foam) were synthesized and impregnated with the active metal species, labeled as catalysts D, E, and F, respectively. They were tested for catalyzing n-butane oxidative dehydrogenation. The results obtained are presented in Table 7. The activity of the catalysts is in the order of catalyst E<D=F. The conversions were improved compared to the conventional silica supported catalysts. Dehydrogenation products selectivity was almost similar among the three catalysts. However, the selectivity increased slightly by going from catalysts E through D and F. While selectivity of oxygenate and cracked products showed an opposite trend. Partial oxidation selectivity increased by going from catalysts F through D to E. Butadiene selectivity was slightly higher for catalyst D, which might relate to the catalyst selective 2^{nd} step dehydrogenation ability as shown in a small amount of 1-butene desorbed into the gaseous phase.

TABLE 7
Comparison of catalytic performance for mesoporous SiO₂ support
species in Ni—Bi—O catalyst: 20 wt % Ni-30 wt % Bi—O/support.
Reaction condition: 450° C., O₂/n-C₄H₁₀ = 2.0

| | Catalyst | | |
|---|---|---|---|
| | D | E | F |
| | Support | | |
| | SBA-15 | MCM-41 | SiO₂ foam |
| PD[nm] | 4.5 | 3.1 | 16.4 |
| n-C₄H₁₀ conversion [%] | 28.9 | 18.0 | 29.2 |
| (O₂ conversion) | (57) | (27) | (55) |

Selectivity*¹ [C %]

| | | | |
|---|---|---|---|
| DH | 75.2 | 74.6 | 77.6 |
| 2-C₄H₈ | 15.4 | 20.5 | 17.2 |
| 1- C₄H₈ | 12.3 | 14.0 | 14.8 |
| BD | 47.5 | 40.1 | 45.6 |
| OC | 19.7 | 18.8 | 21.0 |
| PO | 5.1 | 6.6 | 1.4 |
| (1-C₄H₈ + BD)*² | 59.8 | 54.1 | 60.4 |
| BD/(1-C₄H₈ + BD) %*³ | 79.4 | 74.1 | 75.4 |
| BD yield | 13.7 | 7.2 | 13.3 |

*¹DH: dehydrogenation, BD: butadiene, OC: oxygenate and the cracked, PO: partial oxidation.
*²selectivity at the 1^{st} step dehydrogenation.
*³selectivity at the 2^{nd} step dehydrogenation.

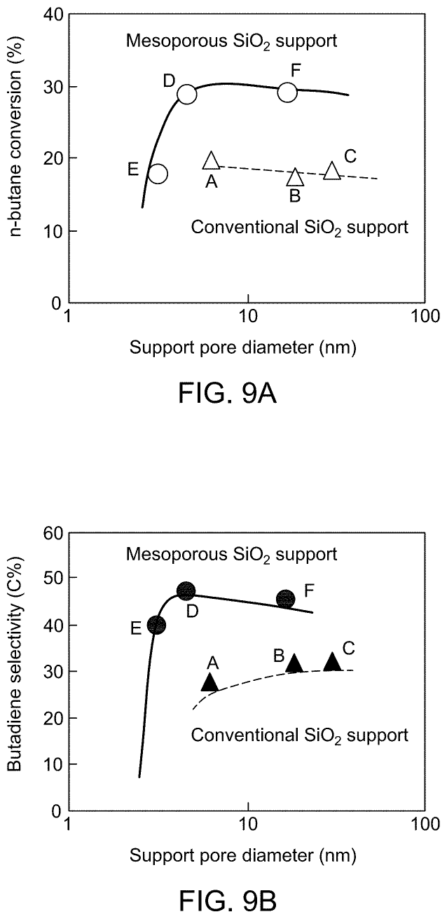

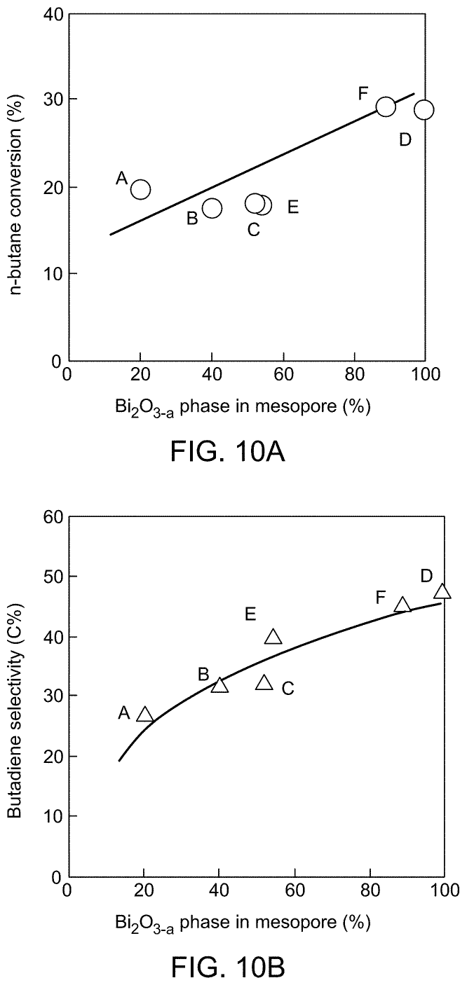

It was reported that the dispersion of active species, their reducibility, and catalytic performance are effectively controlled by the pore sizes of both the conventional and mesoporous supports [D. Song, J. Li, J. Mol. Catal. A: Chem. 247 (2006) 206, incorporated herein by reference in its entirety]. In our case, the effect of support pore diameter on the main catalytic performances (n-butane conversion and butadiene selectivity) showed a completely different trend between mesoporous silica and conventional silica, as shown in FIGS. 9A and 10B. Catalytic activity and butadiene selectivity of the mesoporous silica showed a clear superiority over the conventional silica even at similar pore diameter region. Therefore, the superiority is considered not only due to the pore size or the porosity but also owing to the ordered silica structure.

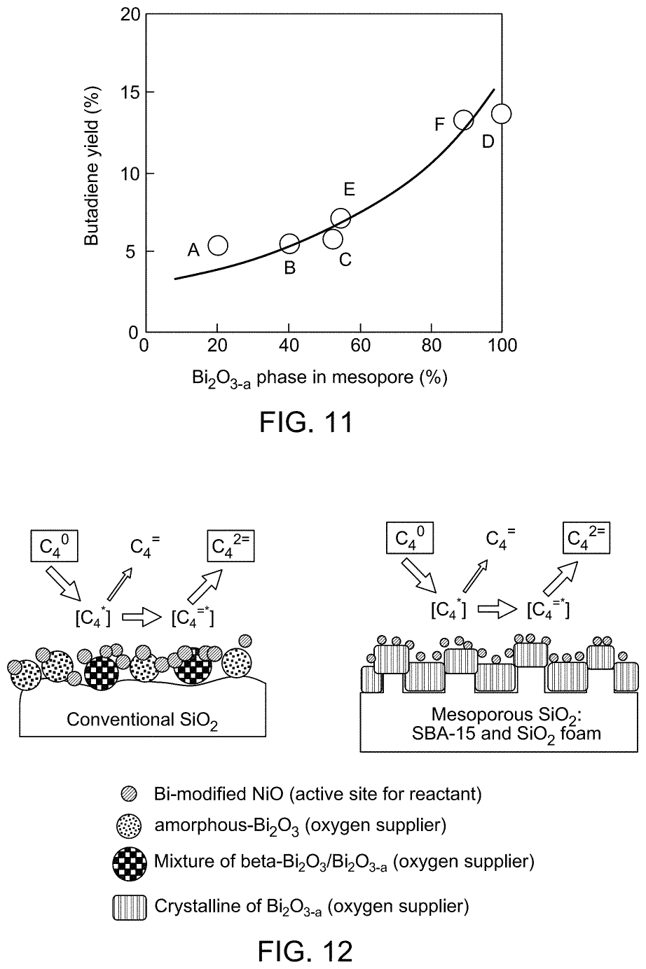

The peak intensity of the Bi₂O₃₋ₐ (a=0.2-0.4) phase has been correlated to the n-butane conversion and butadiene selectivity as presented in FIGS. 10A and 10B. For both conventional and mesoporous silica supported catalysts, an increase in the concentration of the Bi₂O₃₋ₐ phase has enhanced both the conversion and selectivity. This is an indication that the phase serves as an active phase for dispersing NiO which is active and selective for dehydrogenation for butadiene formation. The trends of conversion and butadiene selectivity are indicated by corresponding butadiene yield as shown in FIG. 11. It is clear that the Bi₂O₃₋ₐ phase in mesopore strongly affects butadiene yield.

### Example 5

### Modelling of Reaction and Catalyst

### Role of Support in Catalyst Preparation

The active species of NiO and BiOₓ interact and disperse differently in different mesoporous silica supports. This interaction generates different active sites and active oxygen supplier for a continuous redox cycle of the active species. The effect of mesoporous silica support is important to NiO on hierarchical NiO—Bi₂O₃₋ₐ nanoparticles cohabitation. The SBA-15 support works with the NiO species to produce Bi₂O₃₋ₐ, and high dispersion of NiO.

Model of Support Effect on Butadiene (C₄⁻) Selectivity

FIG. 12 shows a schematic representation of the effect of silica support on the selectivity to butadiene. Conventional SiO₂ supported catalysts mainly have a mixture of beta-Bi₂O₃ and Bi₂O₃₋ₐ as active oxygen supplier, hence the catalyst show an overall high dehydrogenation selectivity but low 1^{st} and 2^{nd} step dehydrogenation selectivity to butadiene. This resulted in relatively high 1-butene desorbed into the gaseous phase, because 2^{nd} step dehydrogenation selectivity requires moderate and strong base more than 1^{st} step dehydrogenation selectivity. Mesoporous silica supported catalysts have a highly crystalline Bi₂O₃₋ₐ phase which acts as active and selective oxygen supplier as well as selective active site (moderate and strong base) for butadiene production. As a result, the catalysts show very high 2^{nd} step dehydrogenation selectivity and improved overall butadiene selectivity.

### Example 6

Ni—Bi—O/structurally mesoporous (MCM-41, SBA-15, and foam) SiO₂ catalysts showed high catalytic performance for oxidative dehydrogenation of n-butane to butadiene. The mesoporous SiO₂ supports have a clear superiority in the activity and selectivity over conventional SiO₂ supports. The butadiene selectivity of catalysts supported on different silica is ranked in the order: SBA-15>SiO₂ foam>MCM-41>conventional SiO₂. The performance of catalyst may be related to the degree of Bi₂O₃₋ₐ formation by template effect of structurally porous SiO₂. This effect is accelerated by NiO co-impregnation. SBA-15 catalyst with the best catalytic performance (butadiene selectivity of 47.5% at n-butane conversion of 28.9%) possessed 99.5% of the bismuth oxides having a Bi₂O₃₋ₐ phase, which was oriented along the SBA-15 silica lattice due to the silica chain template effect. NiO redox property with an increased moderately reducible part, and an improved acid/base property containing both moderate/strong basic sites and weak/moderate acid sites without strong acid sites reflected an enhanced phase behavior at the order of amorphous Bi₂O₃<alpha/beta-Bi₂O₃<Bi₂O₃₋ₐ. The support effect on the catalytic performance was directly resulted from the BiOₓ phase and the hierarchical cohabitation of the Bi—Ni—O active sites.

1: A catalyst, comprising:
a mesoporous silica support which is at least one selected from the group consisting of SBA-15 and mesoporous silica foam; and
a catalytic material comprising nickel oxide and bismuth oxide impregnated on the mesoporous silica support;
wherein the nickel and bismuth atoms of the nickel oxide and the bismuth oxide are present in amounts of 2-30 wt % and 5-40 wt %, each relative to a weight of the mesoporous silica support.

2: The catalyst of claim 1, wherein the mesoporous silica support has a pore volume of 0.2-3 cm³/g and a BET surface area of 200-1,000 m²/g.

3: The catalyst of claim 1, wherein the mesoporous silica support is SBA-15.

4: The catalyst of claim 1, wherein 50-99.9 wt % of the bismuth oxide is present as a non-stoichiometric bismuth oxide relative to a total weight of the bismuth oxide.

5: The catalyst of claim 4, wherein the non-stoichiometric bismuth oxide has a formula of Bi₂O₃₋ₓ, in which x ranges from 0.2 to 0.4.

6: The catalyst of claim 1, which has an average pore diameter of 2-20 nm.

7: The catalyst of claim 1, which has a pore volume of 0.2-2 cm³/g.

8: The catalyst of claim 1, which has a BET surface area of 200-700 m²/g.

9: A method of preparing the catalyst of claim 1, the method comprising:
mixing the mesoporous silica support with an aqueous solution comprising a nickel salt and a bismuth salt to form a mixture;
drying the mixture to form a dried mass; and
calcining the dried mass in air at a temperature of 300-700° C. thereby producing the catalyst.

10: The method of claim 9, wherein a weight ratio of the mesoporous silica support to the nickel salt ranges from 1:3 to 3:1, and a weight ratio of the mesoporous silica support to the bismuth salt ranges from 1:2 to 4:1.

11: The method of claim 9, wherein the aqueous solution comprises the nickel salt at a concentration of 20-80 mM, and the bismuth salt at a concentration of 10-40 mM.

12: A process of oxidatively dehydrogenating n-butane to form butadiene and butenes, the process comprising:
flowing a feed mixture comprising n-butane and an oxidant through a reactor loaded with the catalyst of claim 1, thereby forming butadiene and butenes.

13: The process of claim 12, wherein a molar ratio of the oxidant to n-butane is in a range of 0.1:1 to 8:1.

14: The process of claim 12, wherein the feed mixture is flowed at a flow rate of 10-100 mL/min.

15: The process of claim 12, wherein flowing is performed at a temperature of 300-700° C.

16: The process of claim 12, wherein flowing is performed at a pressure of 35-350 kPa.

17: The process of claim 12, wherein the oxidant is O₂.

18: The process of claim 12, wherein the feed mixture further comprises an inert gas.

19: The process of claim 12, which has a butadiene yield of 7-40 wt % relative to a weight of n-butane.

20: The process of claim 12, which has a molar ratio of butadiene to butenes in a range of about 1:1 to about 4:1.
